(19) United States
(12) Patent Application Publication
(10) Pub. No.: US 2002/0034827 A1
(43) Pub. Date: Mar. 21, 2002

(54) Methods for solid phase nanoextraction and desorption

(76) Inventors: Rajendra Singh, San Jose, CA (US); Remy Cromer, San Jose, CA (US); Michael J. Natan, Los Altos, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/920,440

(22) Filed: Aug. 1, 2001

Related U.S. Application Data
(63) Continuation-in-part of application No. 09/688,063, filed on Oct. 13, 2000.
(60) Provisional application No. 60/222,214, filed on Aug. 1, 2000.
(60) Provisional application No. 60/238,181, filed on Oct. 5, 2000.
(60) Provisional application No. 60/239,662, filed on Oct. 12, 2000.
(60) Provisional application No. 60/265,790, filed on Feb. 1, 2001.
(60) Provisional application No. 60/266,146, filed on Feb. 2, 2001.

(51) Int. Cl. G01N 1/18; G01N 33/551

(52) U.S. Cl. 436/177; 436/524

(57) ABSTRACT

Methods for and materials for separation and analysis of complex materials, including biological materials, are discussed.

### RELATED APPLICATIONS

This application is a continuation-in-part of U.S. patent application Ser. No. 09/688,063, filed Oct. 13, 2000, entitled “Methods for Solid Phase Nanoextraction and Desorption,” and claims the benefit of that application, incorporated herein in its entirety by reference.

This application also claims the benefit of U.S. Provisional Application Ser. No. 60/222,214, filed Aug. 1, 2000, entitled Combinatorial Separation of Biological Material;” U.S. Provisional Application Serial No. 60/238,181, filed Oct. 5, 2000, entitled, “Methods for Solid Phase Nanoextraction and Desorption;” U.S. Provisional Application Serial No. 60/265,790, filed Feb. 1, 2001, entitled “Methods for Solid Phase Nanoextraction and Desorption;” and U.S. Provisional Application Serial No. 60/266,146, filed Feb. 2, 2001, entitled “Nanoparticle-Based Solid Phase Extraction Coupled with MALDI-MS Detection in Proteomic Applications,” all incorporated herein in their entirety by reference.

### FIELD OF THE INVENTION

The present invention relates generally to the separation and analysis of complex materials, including biological materials. More particularly, the present invention relates to methods for the multiplexed separation and/or characterization of components of complex biological mixtures using solid phase extraction techniques, preferably on a micro- or nanoscale. In some preferred embodiments, the present invention employs extraction probes comprising differentiable nanoparticles and combinatorially derived extraction phases.

### BACKGROUND OF THE INVENTION

A variety of methods have been developed for the separation of mixtures for analysis (e.g., filtration, chromatography, extraction, electrophoresis, etc.). However, these methods have not proven sufficient for the separation of biological samples (e.g., blood, plasma, serum, synovial fluid, cerebrospinal fluid, saliva, tears, bronchial lavages, urine, stool, excised organ tissue, bone marrow, etc.). Such samples are comprised of a complex and heterogeneous mixture of molecular and cellular material in which certain components may be quite abundant, while others are present in only trace amounts. The separation and analysis of these types of samples have presented challenges to scientists using conventional techniques.

The currently preferred method for performing proteome analysis (“proteomics”) uses two-dimensional (2-D) gel electrophoresis to separate complex protein mixtures. After electrophoresis and staining, the revealed spots of the gel are excised. The protein is then separated from the gel and typically subjected to enzymatic digestion. The resulting peptide fragments are then characterized by mass spectrometry, such as Matrix-Assisted Laser Desorption Ionization Time-of-Flight (MALDI-TOF MS) or electrospray ionization (ESI MS). The original protein structure can be reconstructed by matching the identified peptide masses against theoretical peptide masses for known proteins that can be found in protein sequence databases, such as SWISS-PROT. Shortcomings of this technology include the lack of reproducibility of the 2-D gel process, difficulties in protein quantitation, and sample loss when recovering the protein from the gel. 2-D gels also suffer from a separation bias against proteins (and other molecules) of very low and very high molecular weight, and against proteins with the same pI. Accordingly, 2-D gels cannot be used for profiling small organic molecules, chemokines, metabolites, and so on. Many molecules known to be important in various disease states (e.g., cholesterol, thyroid hormone, etc.) are, therefore, not detectable by this method.

Specific affinity binding is a technique used to capture specific target ligands from complex mixtures such as biological fluids. For example, monoclonal or polyclonal antibodies may be immobilized on a surface. When the surface is contacted with the sample, the antibodies bind to components of the mixture. Analysis is conventionally carried out via competitive binding, or in a “sandwich” assay using a secondary antibody. In both modes, there is usually a tag (enzyme, radiolabel, fluorophore, etc.) that is used for detection and/or amplification. An alternative approach is direct detection of bound analytes by surface plasmon resonance or quartz crystal microgravimetry. Specific affinity binding techniques have been applied to proteomics in order to characterize gene products. Although it is highly specific, such immunoseparation has many of the same drawbacks as other assays that take place in two dimensions. Moreover, immunoseparation fails when there is no high-affinity antibody available to components in the sample, which is often the case. In particular, immunoseparation provides unsatisfactory results with respect to (i) unknown molecules, (ii) known protein molecules that are post-translationally modified at or near the high affinity epitope; and (iii) molecules too small to elicit a strong immune response.

One specific affinity binding approach to proteomics where the analysis is limited to known proteins (i.e., proteins for which antibodies are commercially available) is the state-of the-art FlowMetrix system developed and commercialized by Luminex Corp. (Austin, Tex.). The FlowMetrix system uses microspheres as the solid support for performing multiplexed immunoassays. Currently Luminex offers 64 different bead sets. Each bead set can, in principle, support a separate immunoassay and the beads are read using an instrument similar to a conventional flow cytometer. A major limitation of the Luminex approach is that the frequency space of molecular fluorescence used both for microsphere tagging and detection is not wide enough to accommodate nearly as many different assays as would be desirable to fully realize the advantages of multiplexing.

Solid phase micro-extraction (SPME) is a separation technique that combines sampling and analyte concentration. The basic process of solid phase extraction involves absorption of one or more target analytes from a sample matrix into a “solid” extraction phase. During the extraction, exposure of the extraction phase to the sample leads to the partitioning of analyte between the sample and extraction phase. The amount of any particular analyte that is extracted from the sample depends on a number of factors, including the partition coefficient.

A device for performing SPME was the subject of U.S. Pat. No. 5,691,206, entitled, “Method and Device for Solid Phase Microextraction and Desorption,” incorporated herein in its entirety by reference. As described therein, a thin coat of polymer or other extraction phase is coated on a fused silica fiber. The coated fiber is contained within a hollow needle extending from the barrel of a syringe-like apparatus and can be extended or retracted using a plunger. To extract analytes from a sample, the needle is inserted into the sample and the coated fiber is extended, exposing the extraction phase to the sample matrix containing the analytes. The sample matrix can be a gaseous sample, a liquid sample, or even the headspace above a liquid sample. After the micro-extraction has been allowed to take place, the fiber is retracted and the needle is removed from the sample. The extracted analytes can then be delivered to a suitable instrument for analysis.

SPME has been successfully coupled to high pressure liquid chromatography (HPLC) and gas chromatography (GC). For analysis by mass spectrometry (MS), analytes adsorbed into the extraction phase may be thermally desorbed and studied by MALDI-MS or Surface Assisted Laser Desorption Ionization mass spectrometry (SALDI-MS), or the analytes may be ionized by electrospray techniques.

SPME has been used for numerous applications in pharmaceutical science, environmental science, biological science, and chemical science. In theory, SPME has the potential to be used for any application in which chromatographic separation is desired. In many contexts, SPME is simpler and faster than traditional solvent-solvent extractions, and produces extracts of equal or greater purity. SPME has been successfully used, for example, to extract pyrazines from peanut butter, fatty acids from milk, and amphetamines from biological fluids.

As it is currently practiced, however, SPME has several important limitations. First, performing SPME using a single fiber does not allow for multiplexing. The single needle method described in the literature would be of limited value for larger scale efforts that require many experiments to be carried out simultaneously or in rapid succession in the same sample. It would be impracticable, for example, for a full-scale proteomics effort to rely on existing SPME techniques.

Second, the small number of solid extraction phases currently available necessarily limits SPME's selectivity as a separation technique. In the original SPME literature, the extraction phase associated with the fiber probe was polydimethylsiloxane (PDMS) or polyacrylamide (PA). These materials possess the fundamental properties necessary to effect SPME—they are chemically stable, they are able to be cast as a thin film, they have a semi-porous or porous geometry, and they have a reasonably high affinity for one or more classes of molecules. In particular, PDMS has a high affinity for non-polar organics and PA has a high affinity for polar organics. However, neither material exhibits particularly high affinity for water-soluble species. Efforts to increase the number and selectivity of SPME extraction phases have met with only limited success—there are now roughly ten different commercially available extraction phases for use in SPME. However, considering the diversity of structure present in the proteome, as well as in the roughly 10,000 different low molecular weight species known to be present in blood, it is clear that SPME in its current method of practice—using single needle extractions and a small number of different extraction phases—is not well-suited for comprehensive profiling of biological samples.

In some cases, researchers have resorted to using two or more different separation methods in tandem in order to profile complex mixtures; for example, liquid chromatography followed by mass spectrometry (LC-MS). However, such “hyphenated” separation techniques generally require increased sample volume and have been hampered by incompatibilities with respect to different separation techniques and the methods eventually used to analyze the separated analytes.

Superimposed on the challenges presented using conventional techniques to analyze biological samples is the pressure to do so faster and with smaller sample sizes. Indeed, advances in medicine and biology have resulted in a paradigm change in what is traditionally defined as bioanalytical chemistry. A major focus of new technologies is to generate what could be called “increased per volume information content.” This term encompasses several approaches, from reduction in the volume of sample required to carry out an assay, to highly parallel measurements (“multiplexing”), such as those involving immobilized molecular arrays, to incorporation of second (or third) information channels, such as in 2-D gel electrophoresis or CE-electrospray MS/MS. It also encompasses efforts to achieve miniaturization of the machinery of analysis—as in Bio-Micro-Electro-Mechanical Systems (Bio-MEMS), microfabricated devices using silicon, glass and polymer substrates that have been used in electrophoresis, electrochemistry and chromatography to reduce sample volume and increase speed and throughput. See, e.g., Manz, A., Becker, H. Eds., “Microsystem Technology in Chemistry and Life Science,” Springer-Verlag: Berlin (1998).

Unfortunately, many of these seemingly revolutionary technologies are limited by a reliance on relatively pedestrian materials, methods, and analyses. For example, the development of DNA microarrays (“gene chips”) for analysis of gene expression and genotyping by Affymetrix, Inc. (Santa Clara, Calif.), Incyte Genomics, Inc. (Palo Alto, Calif.) and others provides the wherewithal to immobilize up to 20,000 different fragments or full-length pieces of DNA in a spatially-defined 1 cm² array. At the same time, however, the use of these chips in all cases requires hybridization of DNA in solution to DNA immobilized on a planar surface, which is marked both by a low efficiency of hybridization (especially for cDNA) and a high degree of non-specific binding. It is unclear whether these problems can be completely overcome.

A second example of how groundbreaking techniques can be slowed by inferior tools, is in pharmaceutical discovery by combinatorial chemistry. Solution phase, 5 to 10 μm diameter latex beads are used as sites for molecular immobilization in some protocols. Exploiting the widely adopted “split and pool” strategy, libraries of upwards of 100,000 compounds can be simply and rapidly generated. As a result, the bottleneck in drug discovery has shifted from the synthesis of candidates to screening, and equally importantly, to compound identification (i.e., knowing which compound is on which bead). Current approaches to the latter problem include “bead encoding”, whereby each synthetic step applied to a bead is recorded by the parallel addition of an organic “code” molecule. Reading the code allows the identity of the drug lead on the bead to be identified. Unfortunately, the “code reading” protocols are far from optimal. In such strategies, the code molecule must be cleaved from the bead and separately analyzed by HPLC, mass spectrometry or other methods. In other words, there is at present no way to identify the large number of potentially interesting drug candidates by direct, rapid interrogation of the beads on which they reside, even though there are numerous screening protocols in which such a capability would be desirable.

Two alternative technologies with potential relevance both to combinatorial chemistry and genetic analysis involve “self-encoded beads”, in which a spectrally identifiable bead substitutes for a spatially defined position on a solid supporting chip. In the approach pioneered by Walt and co-workers, beads are chemically modified with a ratio of fluorescent dyes intended to uniquely identify the beads, which are then further modified with a unique chemistry (e.g., a different antibody or enzyme). The beads are then randomly dispersed on an etched fiber array so that one bead associates with each fiber. The identity of the bead is ascertained by its fluorescence readout, and analytes are detected by fluorescence readout at the same fiber in a different spectral region. The seminal reference (Michael et al., Anal. Chem., 70, 1242-1248 (1998)) describing this technology suggests that with 6 different dyes (15 combinations of pairs) and with 10 different ratios of dyes, 150 “unique optical signatures” could be generated, each representing a different bead “flavor.” A very similar strategy is used by Luminex that combines flavored beads ready for chemical modification with a flow cytometry-like analysis. (See, e.g., McDade et al., Med. Rev. Diag. Indust., 19, 75-82 (1997)). Luminex states that its self-encoded beads enable researchers to assay up to 100 analytes in a single sample. The particle flavor is determined by fluorescence and, once the biochemistry is put onto the bead, any spectrally distinct fluorescence generated due to the presence of analyte can be detected. As currently configured, it is necessary to use one color of laser to interrogate the particle flavor, and another, separate laser to excite the bioassay fluorophores.

A significant limitation of self-encoded latex beads is that imposed by the wide bandwidth associated with molecular or nanoparticle-based fluorescence. If the frequency space of molecular fluorescence is used both for encoding and for bioassay analysis, it is hard to imagine how, for example, up to 20,000 different flavors could be generated. This problem may be alleviated somewhat by the use of semiconductor nanocrystals (“quantum dots”), which exhibit narrower fluorescence bandwidths. (See, e.g., Bruchez et al., Science, 281, 2013-2016 (1998)). If, however, it were possible to generate very large numbers of intrinsically-differentiable particles by some means, then particle-based bioanalysis would become exceptionally attractive, insofar as a single technology platform could then be considered for the multiple high-information content research areas, including combinatorial chemistry, genomics, and proteomics (via multiplexed immunoassays).

Surface derivatized probes consisting of self-assembled monolayers (SAMs) terminated with ionic functional groups also have been used for extracting peptides/proteins. (Warren et al., Anal. Chem., 70, 3757-3761 (1998)).

SPME followed by CE as the second dimension has been used to analyze a mixture of peptides from a proteolytic digest. (Yates, Anal. Chem., 71, 2270-2278 (1999)). Although the SPME-CE/MS improved the concentration detection limit by more than two orders of magnitude when compared to CE-MS alone, the large electro-osmotic force of the aminopropylsilane (APS) coated capillary tended to elute all the peptides in a relatively short period of time. This presents the possibility of confounding results owing to the co-elution of compounds.

A strategy has been used for the separation of MHC class I peptides, several thousand peptides at sub-femtomolar concentrations. The literature reports immuno-affinity concentration followed by reverse phase, and subsequent concentration on specially designed membranes capillaries. (Naylor, Chromatogr., 744, 237-78 (1996)). In addition, a comprehensive two-dimensional separation technique has been described for profiling proteins. (Jorgenson et al., Anal. Chem., 69, 1518-1524 (1997)).

There is a need for analytical methods of high sensitivity and selectivity that have the power to resolve and profile different components of a complex mixture, such as a biological fluid. At the same time, there is a need for such methods to be able to identify and quantitate minute quantities of biomolecules in small sample sizes, potentially even in single cells.

There is also a need for streamlined and automated methods for analyte capture that are compatible with sophisticated separation and detection technologies, such as HPLC, GC, CE, and MS.

In addition, there is also a need for methods for rapidly interrogating a biological sample that can be multiplexed. In particular, there is a need to have methods for separation and analysis of low-molecular weight organic molecules, peptides, and larger proteins simultaneously in a microvolume samples.

There is a need for combinatorially-derived extraction phases to extract analytes from a sample. In particular, there is a need for such surfaces that can be used in multiplexed analyses.

### SUMMARY OF THE INVENTION

The present invention relates generally to methods for multiplexed separation and analysis of biological materials. More particularly, the present invention relates to methods for multiplexed characterization of components of biological fluids utilizing solid phase extraction, preferably on a micro-scale or nano-scale. The solid phase extraction methods of the present invention are accomplished using solid supports that have been coated or are otherwise associated with an extraction phase. In some preferred embodiments, the present invention relates to methods and materials for performing solid phase extraction and analysis using nanoparticles that have been coated or are otherwise associated with an extraction phase to extract analytes from a sample. In other preferred embodiments, the solid supports for the extraction phases are arrays of fibers. In some preferred embodiments, the extraction phases are combinatorially derived. The present invention may be used to study normal biological functions, disease, disease progression, and changes associated with virtually any perturbance to the organism. Indeed, the present invention provides information that may be analyzed to identify biological markers that can be measured and evaluated as indicators of normal biological processes, pathogenic processes, or pharmacologic responses to a therapeutic intervention.

The present invention includes methods for performing solid phase extraction in which particles are used as the solid support. In preferred embodiments, such particles are differentiable from one another. In some preferred embodiments, the particles are encoded nanoparticles that allow extremely high-level assay multiplexing in solution, essentially combining the advantages of arrays (e.g., gene and/or protein chips) with the advantages of solution-based assays. Encoded nanoparticles may be used according to the present invention to simultaneously perform thousands of chemically and biochemically selective solid phase nano-extractions (SPNE) on samples, and then interface with a means for analyzing the extracted molecules, including mass spectrometry and/or fluorescence. In highly preferred embodiments, the encoded nanoparticles are rod-shaped nanoparticles whose composition varies along the length of the rod (also known as Nanobarcodes™ identification tags). Although not necessary to achieve the benefits of the present invention, such segmented-nanoparticles increase the power of the analytical separation methods described herein.

In some preferred embodiments, the present invention includes methods of carrying out solid-phase nano-extractions (SPNE); for example, when nanoparticles are used. Such methods preferably employ particles that are distinguishable from one another. Other solid supports within the scope of the invention include beads and fibers. Also included within the scope of the invention are methods for the simultaneous use of a plurality of differentiable solid supports, each associated with a different extraction phase for solid phase extraction.

### BRIEF DESCRIPTION OF THE FIGURES

FIG. 1 is a schematic diagram illustrating multiplexed solid-phase nano-extraction using encoded nanoparticles, according to a preferred embodiment of the present invention.

FIG. 2 schematically illustrates the preparation of segmented-nanoparticle extraction probes containing self-assembled monolayer extraction phases.

FIGS. 3A-3E show five different classes of extraction probes prepared according to the method of FIG. 2.

FIG. 4A is a mass spectrum of a low-molecular weight fraction of plasma.

FIGS. 4B-4F are mass spectra of different extracted analytes from the plasma sample of FIG. 4A following extraction by the illustrated extraction probes.

FIG. 5 illustrates the preparation of a library of gamma-hydroxy amides of dextran coated segmented-nanoparticles.

FIG. 6 illustrates the preparation of streptavidin-coated segmented-nanoparticles.

### DETAILED DESCRIPTION OF THE INVENTION

The present invention is directed to novel separation and analytical methodologies. In particular, the present invention is directed to methods for separation and analysis that can be multiplexed. Most generally, multiplexing refers to multiple measurements performed on the same sample. These measurements can be carried out simultaneously or in rapid succession; preferably, they are carried out simultaneously. These measurements may be carried out in a single sample aliquot, or in a divided sample; preferably, the measurements are carried out in the same sample volume. Thus, multiplexing covers the range from multiple measurements taken simultaneously in a single sample, to multiple measurements carried out in different locations on a single sample (e.g., a gene chip) to multiple measurements carried out in succession on different sample aliquots. The common thread of multiplexing is multiple measurements on a sample.

Measurement, as used herein, may refer to any information about a sample, any operation performed on said sample mixture, e.g., the separation of components, the differential or non-differential concentration of analytes, or determining the existence and/or quantity of any analyte or class of analytes within such sample mixture. The methods of the present invention utilize extraction probes comprised of solid supports that are partially or totally coated, physically or chemically attached to, or in some way associated with an extraction phase.

In certain preferred embodiments the extraction probes of the present invention are uniquely derivatized nanoparticles. Such nanoparticle extraction probes may be introduced into the sample where they can independently assort in three-dimensions, allowing the extraction phase associated with each nanoparticle to contact and interact with analytes present in the sample. Once retrieved from the sample the nanoparticle probes, and absorbed analytes, can be directly interrogated via analytic methods. The retrieval may be accomplished by any means, including filtration, centrifugation, magnetic means, or self-assembly. As described more fully below, the method of the present invention is rapid and can be automated and multiplexed; in addition, the incorporation of the extraction phase with the nanoparticles also may be accomplished on an automated basis, using combinatorial methods to synthesize the extraction phase.

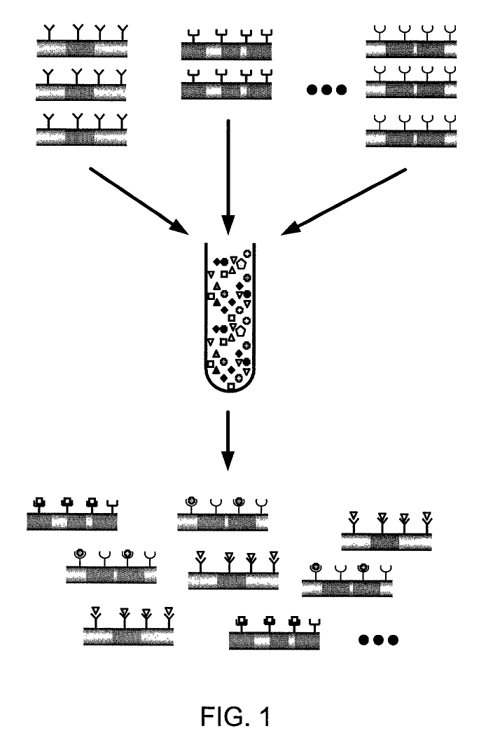

FIG. 1 schematically illustrates such a method of the invention for performing solid-phase nanoextraction using coded and derivatized nanoparticles. A collection of differently coded extraction probes, each containing a solid support and an extraction phase, is contacted with a sample containing a variety of analytes. As indicated by the different shapes of the extraction phase, each code of the solid support corresponds to a unique extraction phase and is therefore capable of extracting a different analyte (or class of analyte). During contact between the sample and probes, the probes interact with certain of the analytes. After sufficient contact, the extraction probes are separated from the sample to extract the associated analytes. As discussed further below, subsequent analysis of the extracted probes can include differentiating among or separating the different types of extraction probes, or detecting, identifying, or quantifying the analytes extracted by the probes.

In other preferred embodiments, the extraction probes are arrays of derivatized fibers. The fibers can be sized in the range of 100 μm diameter or, preferably, they can be of less than 1 micron diameter. Configured as arrays, the fibers (or “needles”) can be coated or otherwise associated with extraction phases and exposed to the sample in a multiplexed fashion that lends itself to automation. The nature of the associated extraction phase can be readily identified by the spatial address of the fiber. Furthermore, presentation to a suitable analytic instrumentation is also facilitated when the extraction probe needles are configured in arrays.

In other preferred embodiments, the extraction probes may be beads. The beads can be readily derivatized with extraction phases.

In some preferred embodiments, the extraction phase is comprised of combinatorially-derived materials (e.g., those derived from a split/pool synthesis). (See Schultz et al., U.S. Pat. No. 5,985,356, entitled, “Combinatorial Synthesis of Novel Materials” and Wu et al., U.S. Pat. No. 6,045,671, entitled, “Systems and methods for the combinatorial synthesis of novel materials,” both incorporated herein in their entirety by reference). The combinatorially-derived extraction phase is preferably a polymer. However, there are a number of other materials (e.g., inorganic materials, metal alloys, oxides, glasses, ceramics, zeolites, polyelectrolyte multilayers) or combinations thereof, that may be useful as combinatorially-derived extraction phases. Furthermore, the combinatorially-derived extraction phase may be generated randomly, synthesized in a more controlled manner, or chosen from available materials. For example, a series of extraction phases chosen from the available chromatographic literature is contemplated as being “combinatorially” derived. Likewise, a single extraction phase, used in different ways (i.e., at different densities, porosities, pH values, etc.) is contemplated as being “combinatorially” derived. In this way, a library of combinatorially-derived extraction phases may be generated, selected, purchased, prepared or obtained by one means or another, such that each member will differ (to a greater or lesser extent) from the others, e.g., by their physical, chemical or functional properties. The various combinatorially-derived surfaces can be used to increase the selectivity of separation and analytical methodologies.

According to the present invention, the extraction probes are comprised of solid supports coated or otherwise associated with an extraction phase. In those embodiments of the invention comprising an array of extraction probes, or collection of encoded extraction probes, each solid support may be associated with an extraction phase that may interact differently with the sample and any analytes contained therein. Much of the power of the present invention arises from multiplexing—hundreds or even thousands of these discrete separations can be accomplished simultaneously.

For example, assume a sample contained only three analytes, A, B and C. Extraction probe I is comprised of an extraction phase that will extract a quantity of analyte A from the sample, but will not extract any of analytes B or C. Extraction probe II comprises an extraction phase that extracts a quantity of analytes A and B from the sample, but not analyte C. Extraction probe III, however, only extracts analyte C. By the use of multiplexing, e.g., simultaneously contacting the sample with extraction probes I, II and III, one can detect for the presence of analytes A, B and C. By expanding this example to, for example, a system whereby 10,000 different extraction probes are utilized, it can be shown how a complex sample, such as a biological material, can be profiled. Some analytes within a sample will be extracted by a large number of the extraction probes, while other analytes will only be extracted by a few extraction probes. In addition, it will not just be a question of whether or not the analyte has been extracted—there will be degrees of extraction. By detecting the presence of any analytes extracted from each of the extraction probes, it is possible to profile virtually all of the analytes within a highly complex sample. In some embodiments, the quantity of some or all extracted analytes may be determined. By performing the extraction simultaneously, it is possible to generate a huge amount of information about a sample in any extremely short period of time.

The concentration of analytes via extraction into the extraction phase serves as a discrete separation process. In addition to the types of extraction phases and solid supports empolyed, the extraction can be manipulated by changing the reaction conditions, such as the temperature, pH, pressure, concentration, ionic strength. In addition, the exposure time may be varied. Some analytes can be expected to require more time than others to partition into the extraction phase. In many cases, it is impracticable to wait for equilibrium to be reached. Rather, the extraction probes can be exposed to the sample for a given length of time, and extract the amount of analyte in that time. By varying these conditions, additional information can be obtained from the sample. For instance, using the hypothetical above, assume that the analytes extracted by the probes changes as a function of pH. Thus, at a higher pH, probe III might extract a third analyte, Analyte D. Thus, changing the pH would allow additional information to be obtained using the same set of extraction probes.

In one series of preferred embodiments, the solid supports used to achieve the multiplexed separation and analysis of the present invention are freestanding nanoparticles. By “freestanding” it is meant that nanoparticle solid supports that are produced by some form of deposition or growth within a template have been released from the template. Such particles are typically freely dispensable in a liquid and not permanently associated with a stationary phase. Nanoparticles that are not produced by some form of deposition or growth within a template (e.g., self-assembled segmented-nanoparticles) may be considered freestanding even though they have not been released from a template. The term “freestanding” does not imply that such nanoparticles must be in solution (although they may be) or that the particles can not be bound to, incorporated in, or a part of a macro structure.

In general, such a multiplexed separation and analysis using nanoparticle probes would proceed as follows: The nanoparticles individually are coated or otherwise associated with an extraction phase and then put in contact with the sample. Because of the small size of the nanoparticles, only a small amount of sample is required; in some instances merely a few drops of whole blood. Then, after sufficient time has been allowed for the extraction to take place, the nanoparticles are recovered from the sample. Recovery may be accomplished by any of a number of known means (e.g., centrifugation, filtration, etc.). In some embodiments, the nanoparticle extraction probes can be made magnetic to facilitate recovery. Once recovered, the analytes extracted from the sample can be detected by a suitable analytic device (e.g., a mass spectrometer or fluorescence detector). In certain embodiments, the nanoparticle extraction probe may be delivered to the analytic device by microfluidic means. The analytes may or may not be eluted from the extraction probe before delivery to the analytic device. When desired, elution may be accomplished by of any of a number of means known in the art, including washing with a suitable reagent/solvent.

Coated with combinatorially-derived extraction phases (as described more fully below), the nanoparticles allow a combinatorial separation to proceed on a nanoscale. Thus, nanoparticle-based assays may utilize the combination of solid phase extraction techniques, microfluidics and mass spectrometric techniques, to accomplish differential proteomic and small organic molecule (“orgeomic”) profiling as well as detection of other parameters (e.g., presence of a pathogen) in a biological sample. In a series of preferred embodiments, this may include (i) placing a combinatorial self-assembled monolayer extraction phase on the nanoparticle for the purpose of extraction of any type of molecule or other analyte from a complex biological fluid; and (ii) using a nanoparticle/mass spectrometry interface to analyze the surface adsorbed molecules with nanoparticle specific ESI, MALDI, or matrix-free ionization-time of flight (SALDI-TOF) techniques.

Mass spectrometry is preferentially selected as a detection element for broad-based differential, comprehensive molecular analysis (differential molecular phenotyping) because of its applicability for both high and low molecular weight species. Indeed, it is presently the only technique capable of both furnishing molecular identification of peptide fragments associated with large proteins and molecular weight/identification of molecules in the 100-500 amu range. Moreover, insofar as several masses can be identified simultaneously, mass spectrometry is an inherently multiplexed detection technique.

Mass spectrometry is rapidly becoming the tool of choice for detailed identification and analysis of polypeptides and proteins. There are two widely-used methods for biomolecular sample introduction in mass spectrometry: Electrospray (ESI) and matrix-assisted laser desorption/ionization (MALDI).

Electrospray ionization mass spectrometry (ESI/MS) has gained recognition as an important tool in the study of proteins and protein complexes. In electrospray ionization, the eluent containing the analyte of interest is pumped at high pressure through a hypodermic needle and an electrical potential is applied to the resulting fine spray of particles. As the highly charged droplets vaporize, molecular ions are released and filtered by quadrupoles to the mass detector.

In MALDI, the analyte of interest is embedded into a solid ultraviolet-absorbing organic matrix that vaporizes upon pulsed-laser irradiation, carrying with it the analyte. (See, e.g., Karas et al., Anal. Chem. 60, 2299-2301 (1988)). During this process the energy absorbed by the matrix is transferred to the analyte that is ionized. The gas phase analyte ion is then sent to the Time-Of-Flight (TOF) mass analyzer. MALDI-TOF is currently successfully used for the analysis of proteins, polypeptides and other macromolecules. Even though the introduction of an organic matrix to transfer energy to the analyte has advanced tremendously the field of desorption mass spectrometry, MALDI-TOF still has some limits. For instance, the detection of small molecules is not practical because of the presence of background ions from the matrix. Also, MALDI experiments are inherently sensitive to matrix choice—matrix type as well as matrix amounts must often be tailored to the nature of the analyte (a severe limitation to the analysis of complex mixtures).

More recently, Sunner et al. have introduced the term SALDI for Surface-Assisted Laser Desorption/Ionization (Sunner et al, Anal. Chem. 67, 4335 (1995)). This technique is matrix-free, allows analysis of small organic molecules and yields performances similar to MALDI. Noble metal nanoparticles may be a superior choice for laser-based ionization, for two reasons. First, colloidal noble metal nanoparticles exhibit very large extinction coefficients in the visible and near IR. This contrasts with organic matrices. Second, irradiation of Au nanoparticles is known to lead to dramatic enhancements in electric field strength at the particle surface. This leads to increased ionization efficiencies and is the basis of surface-enhanced Raman scattering. Moreover, combined with the ability to encode segmented-nanoparticles, SALDI-MS becomes a powerful molecular fingerprinting tool.

The technical hurdle in the art associated with mass spectrometry-based approaches to differential molecular analysis concerns sample separation—that is, how to convert exceedingly complex samples containing thousands of species into simpler mixtures containing a dozen or fewer analytes—a problem to which the present invention is directed.

As more fully described below, nanoparticle extraction probes offer an important means to perform solid phase extraction on the nanometer scale, and to perform combinatorial extractions, whereby thousands of chemically distinct extractions are performed simultaneously.

Clearly, the ability to use distinctly coded particles significantly increases the power of the present invention. In particular, it allows identification of the extraction phase used to extract a compound or compounds of interest. While solid phase extraction is out with identical, non-distinguishable extraction probes, it complicates analysis in two significant respects. First, it impairs the ability to match the proper analysis to the proper analyte. To illustrate: Each non-distinguishable solid support in an assembly is coated with one of a number of extraction phases, each extraction phase having a different affinity and/or partition coefficient, and possibly with a different selectivity as well. When these extraction probes are contacted with a complex molecular mixture, each probe “samples” a different fraction of the mixture. In such a circumstance, it is expected that analysis of the material bound to each probe (or some fraction of the probes) subsequently will be performed. Because the probes each capture different types of classes of analyte, the preferred analysis conditions could be quite different. This information is not available using unencoded extraction probes because the extraction phase (and thus the type or class of analyte) is unknown. With encoding, each type (“flavor”) of extraction probe can have a unique extraction phase, and that information is available when the material extracted by the probe is analyzed.

For example, one would use very different ionization conditions (voltage, flow rate, etc.) for ESI mass spectrometric analysis of positively and negatively charged species. However, without the information provided by the encoded extraction probe, such decisions cannot be made or must be made arbitrarily. Thus, even in this very simple case, where positively charged species are captured on one extraction probe, and negatively charged species on another, the use of an encoded extraction probe is a significant advantage. Mass spectrometry literature contains dozens of different analyte-specific ionization conditions, further increasing the value of extraction probe encoding.

A second benefit of extraction probe encoding involves the subsequent use of the information gained following extraction. For example, if analysis of one particular extraction probe leads to the identification of a material of interest, it may be desirable to design an extraction phase that could bind more (or less) of the analyte. In such cases, knowledge of the identity of the extraction phase itself, as could be obtained using encoded extraction probes, is essential. While this is pertinent when identification of the analyte has occurred by a method that furnishes chemical structure information (e.g., mass spectrometry, NMR, Raman, IR), the use of an encoded extraction probe is even more critical if detailed analysis of extracted compounds is not carried out. For example, if all that is known about a particular analyte is that is was partitioned onto an extraction probe and, upon chromatographic or electrophoretic separation (e.g., by GC, LC, or CE) exhibited a certain retention time, the information has limited value—it cannot be repeated, since it was not known from which extraction probe the compound was eluted. More importantly, the experiment cannot be correlated to another experiment, because the specific identity of the extraction probe is unknown.

In contrast, if the extraction probe identity is known, the intensity of the peak at issue from that probe in Sample 1 can be directly compared to the corresponding intensity from that same probe in Sample 2. The ability to compare results from different samples is essential to comprehensive phenotypic analysis via combinatorial separation. Indeed, in such cases, the goal is to detect changes in individual species, or more likely, patterns of changes in species. Thus, the precise identification of every individual compound in a complex mixture, such as plasma, is less important (and less feasible) than a comparative sample to sample analysis. In this case, which is one of the primary applications of combinatorial separations, the use of multiple extraction phases is of very limited benefit without a corresponding mechanism for extraction phase identification, e.g., extraction probe encoding.

Thus, the use of encoded extraction probes allows the separation and analytical processes to be multiplexed. Given the diversity of structure present in the proteome, as well as in the “orgeome”—low molecular weight species (<10,000 Da.)—the greater the number of encoded extraction probes possible, the more utility the probes have for comprehensive profiling. A large number of differentiable nanoparticles as solid supports thus complements the ability of combinatorial chemistry to create an equally large number of materials for inclusion in extraction phases. Such combinatorially-derived extraction phases could simultaneously capture a number of analytes from biological samples on distinctly coded extraction probes.

Such combinatorially-derived extraction phases can be made of any material. In certain preferred embodiments they are polymers. However, they can be composed of any material, e.g., oxides, glasses, ceramics, clays, zeolites, dendimers, oligomers, macromolecular complexes, supramolecular assemblies. To illustrate: A library of synthetic organic polymers could be obtained using combinatorial chemistry techniques, each member having its own particular properties. Then, the encoded nanoparticle solid supports (e.g., Nanobarcodes™ identification tags) could be uniquely coated with one member of the combinatorially-derived polymer library. An assembly or array of nanoparticle extraction probes could be prepared, each having a specific extraction phase and associated properties. The result is a library of unique nanoparticle extraction probes. Because hundreds or thousands of nanoparticle extraction probes can be contacted with even a small sample at the same time, and because it is known (or can be determined) what extraction phase is associated with a given nanoparticle solid support, differential analysis is possible.

Solid phase extraction arrays, such as protein chips, rely on the physical position on the array to identify the matrix of captured analytes on the chip surface. Thus, the nature of the associated extraction phase, for example, can be readily identified by the spatial address of the extraction phase. Such two-dimensional arrays enable assays of multiple analytes to be conducted in parallel. However, slow diffusion of analytes to the planar surface limits its application. Encoded particle-based extraction probes of the present invention provide an alternative approach to in which there is a three-dimensional array in solution (“solution arrays”). Substituting for the spatially defined position on a solid supporting chip array, the encoded information allows individual elements to be addressed when positional information has been eliminated by random distribution in space. This approach retains the advantages of two-dimensional arrays for massively parallel analyses with diverse extraction probes at high throughput, but does not compromise on the kinetics of binding in solution. Furthermore, because each element in a solution array is independent, there is flexibility to interrogate a few or thousands of analytes without the need to fabricate a new chip with a custom set of extraction phases. In addition, a spatially defined position may be introduced to the system of encoded particle-based extraction probes, e.g., after contact with the sample, resulting in an “array of arrays.” This may be accomplished, for example, by sorting the probes into spatially defined areas of a two-dimensional surface; typically, into wells of a microtiter plate. The sorting may occur either before or after contacting the probes with the sample and may be effected by any of a number of means known in the art (e.g., in flow). Reintroducing a spatially defined position will allow the extraction probes to be interrogated in the same ways currently used for two-dimensional arrays (e.g., protein chips). In those embodiments in which a spatially defined position is introduced, it adds another level of multiplexing to the assay—for example, a set of 104 differentiable extraction probes distributed in each well of a 96-well plate will effectively create nearly 10,000 individually addressable extraction probes, and allow nearly 10,000 of extraction phases to be contacted with sample. In this way, using differentiable extraction probes with two dimensional arrays would decrease the cost and time required to obtain useful information, because a smaller set of differentiable solid phases would have to be synthesized.

The analysis of complex mixtures, such as biological fluids, usually involves the removal and concentration of the analytes of interest. Solid supports (e.g., a nanoparticle, bead or fiber) may be associated with different extraction phases for the capture of analytes, such as proteins, organic molecules, or other components of the biologic matrix. The range of extraction phases is broad and can include, without limitation, hydrophobic materials, hydrophilic materials, acids, bases, polyclonal or monoclonal antibodies, aptamers, small molecule receptors, polymers, molecular solids, non-molecular solids, metals, metal ions, cations, and anions, and combinatorial chemistry libraries.

Most require derivatization of the solid support with an extraction phase which has an affinity for a component, or set of components, which may be present in the sample. As described above, the nanoparticle extraction probes are placed in contact with the sample (e.g., mixed) under conditions that allow for analyte capture. The mechanism of capture depends on the nature of the extraction phase. If the extraction phase is, for example, a conventional SPME polymer, the analyte is captured by means of microextraction into the polymer; if the extraction phase comprises an antibody, the analyte is captured by means of specific binding to the antibody.

Thus, for example, a solid support could be derivatized with a thin coating of polydimethylsiloxane (PDMA), an extraction phase that is used to coat the fiber in some conventional SPME applications. When placed in contact with the sample, the PDMA-coated solid support selectively interacts with a set of non-polar organic molecules. Similarly, in another assay, a solid support could be coated with polyacrylamide (PA). Indeed, solid supports coated with conventional SPME polymers would allow a number of extraction phases for concentration of analytes on the extraction probe and delivery of the analytes for analysis.

In preferred embodiments of the invention, an array or assembly of extraction probes is prepared. While in certain embodiments, the members of the array are identical, in preferred embodiments the array is comprised of a plurality of different types of members. Using arrays of extraction probes, such as fiber arrays, or otherwise differentiable extraction probes, such as segmented-nanoparticles (Nanobarcodes™ identification tags), allows the experiments to be performed simultaneously in the same sample. In other words, differentiable extraction probes allow the SPME process to be multiplexed.

The present invention includes imaging and distinguishing between members of arrays or assemblies of nanoparticle extraction probes comprising a plurality of nanoparticle solid supports that are differentiable from each other. The arrays of the present invention can include from 2 to 1×10¹² different and identifiable nanoparticle extraction probes. Preferred assemblies include more than 10, more than 100, more than 150, more than 200, more than 500, more than 1,000 and, in some cases, more than 10,000 different flavors of nanoparticle extraction probes. In highly preferred embodiments, the nanoparticle solid supports that make up the assemblies or collections of probes are segmented. However, in certain embodiments of the invention the particles of an assembly of particles do not necessarily contain a plurality of segments.

In the embodiments of the present invention where the nanoparticles contain some informational content, or where an array of nanoparticles contain a plurality of types of particles, the types of particles are differentiable apart from the nature of the extraction phase associated with each particle type. In this invention, the ability to differentiate particle types or to interpret the information coded within a particle is referred to as “interrogating” or “reading” or “differentiating” or “identifying” the nanoparticle. Such differentiation of particles may be read by any means, including optical means, electronic means, physical means, chemical means and magnetic means. The particle may even contain different sections that will be interrogated or read by different means. For example, one half of a particle may be comprised of segments whose pattern and shapes can be read by optical means, and the other half may be comprised of a segment whose pattern and shapes may be read by magnetic means. In another example, two different forms of interrogation may be applied to an entire particle, e.g., the shape or length of the particle may be read by optical means and the segment patterns by magnetic means.

The wider the range of extraction phases used, the more powerful the separation. By appropriately selecting and designing distinct extraction phases, there is an opportunity to create a large library of different extraction probes. Preparation of the extraction phases by a combinatorial process provides surfaces with varying characteristic extraction phases that will allow extraction of a wide variety of molecules present in the biological sample. Being able to sample a much larger fraction of the number of molecules in a sample would significantly increase the utility of solid phase extraction as an analytic technique. In this way, multiple extractions would occur, either in parallel or in series, each extraction phase targeting some portion of the sample's “molecular structure space.” For example, a set of solid phase extraction experiments could be performed in which each extraction probe is derivatized with an extraction phase comprising one of a spectrum of organic phase polymers, each different in some way from the rest.

This concept can be further illustrated by the following examples. At one extreme, the extraction phases could be highly specific for a certain analyte. Thus, monoclonal antibodies could be used as part of the extraction phase, either bound directly to the solid support or affixed to a polymer on the solid support. In theory, the extraction phase in this instance would exhibit high affinity for one and only one molecule. In real biological systems, however, antibodies to particular proteins will capture the protein of interest as well as any post-translationally modified species for which the modification does not significantly abrogate the antibody-antigen interaction. To capture a large fraction of molecules using this approach would require a large number of parallel or serial experiments, each employing a different antibody. A fundamental limitation of this approach is the finite number of monoclonal antibodies available and—equally significantly—the inability to capture completely new or novel proteins (i.e., those for which no antibodies are available).

At the other extreme, the extraction phase could have a low affinity for a large number of molecules; for example, a C18 reversed phase typically used for HPLC. Here, few species will bind with high affinity, but it will be possible in principle to extract new substances that are present at a sufficiently high concentration, and have a partition coefficient appreciable enough to lead to non-negligible amounts being concentrated in the extraction phase.

In general, pairs of interacting molecules can be exploited in two ways: (1) with an extraction phase to capture a “ligand”, and (2) with an extraction phase to capture a counterligand “receptor.” The table below lists examples of ligands for inclusion in extraction phases to capture/extract specific molecules (counterligands) from biological samples.

| LIGAND | COUNTERLIGAND |
|---|---|
| Cofactors | Enzymes |
| Lectins | Polysaccharides, glycoproteins |
| Nucleic acid | Nucleic acid binding protein (enzyme or histone) |
| Biomimetic dyes | Kinases, phosphatases, Dehydrogenases etc . . . |
| Protein A, Protein G | Immunoglobulins |
| Metals ions | Most proteins can form complexes with metal ions |
| Enzymes | Substrate, substrate analogues, inhibitor, cofactors |
| Phage displays | Proteins, peptides, any type of protein |
| DNA libraries | Complementary DNA |
| Aptamers | Proteins, peptides, any type of protein |
| Antibody libraries | Any type of protein |
| Carbohydrates | Lectins |
| ATP | Kinases |
| NAD | Dehydrogenases |
| Benzamide | Serine Protease |
| Phenylboronic acid | Glycoproteins |
| Heparin | Coagulation proteins and other plasma proteins |
| Receptor | Ligand |
| Antibody | Virus |

Of course, countless other examples of specific interactions are known and may be advantageously used. Ligands used in affinity chromatography, for example, also could be used in extraction phases. However, unlike conventional affinity chromatography, all the extraction probes may be combined in a single tube containing a minimum volume of biological sample. Accordingly, sample size will be greatly reduced compared to currently developed protein arrays. Subsets of extraction probes may bind with high affinity to unknown partners as well; for example, using a library of small molecule ligands in the extraction phase to target a receptor.

It should be noted that from a fundamental perspective, there is no difference between a “partition coefficient,” as the term is used in standard chromatographic and separation science texts, and an “affinity constant,” as that terms is used in bioanalytical work, other than degree. In essence, both the partition coefficient and the affinity constant, in conjunction with other useful (and likewise analogous) parameters such as loading or capture reagent concentration/surface coverage, allow for the accurate prediction of what will happen to a particular molecule in the presence of an extraction phase. Immobilized high-affinity receptors carry out the same chemistry as traditional SPME extraction phases, although with higher affinity for a particular species and with greater selectivity. In certain applications, this increased affinity and selectivity can be advantageous. In other applications, a low affinity, low selectivity extraction phase may be preferred. In still other applications, it will be a combination of high affinity and low affinity, high selectivity and low selectivity extraction phases that can be used to maximum advantage.

For differentiable nanoparticle extraction probes, the highly preferred solid supports are rod-shaped nanoparticles whose composition varies along the length of the rod. These freestanding particles may be referred to as “segmented-nanoparticles” or “Nanobarcodes™ identification tags,” or “nanorods,” though in reality some or all dimensions may be in the micron size range. These segmented-nanoparticles comprise a plurality of segments or stripes which may be comprised of different materials and may be functionalized on selected or all segments. The types of particles are differentiable based on the length, width, shape and/or composition of the particles. This allows a plurality of assays or measurements of analyte concentrations or activities to be performed simultaneously, or in rapid succession, by contacting a solution that may contain said analytes with a plurality of the segmented-nanoparticle extraction probes, wherein each probe comprises an extraction phase (e.g., molecule, species or other material) that interacts with one of said analytes. Like macroscopic bar codes, the probes are “encoded” when the basis for differentiating them (e.g, composition, size, etc.) has informational content. Thus, a certain striping pattern may signify the nature of extraction phase, the origin of the probe, and/or other information).

Besides panning the biological sample for small organic molecules, peptides, and nucleotides, it is possible to take advantage of the segmented-nanoparticle technology to multiplex assays (e.g., immunoassays). The combination of segmented-nanoparticle technology, SALDI and fluorescence based immunoassays into one platform, as described herein, for example, enables the generation of highly sensitive, quantitative, multiplexed immunoassays for known proteins. The ability to merge selectivity, sensitivity, multiplexing, quantitation and mass analysis in the same measurement offers, among other benefits, a minimum of 100-fold increase in sensitivity.

A protocol by which this advance may be achieved is outlined below. First, a specific immunoassay is associated to each “flavor” of segmented-nanoparticle solid support by attaching a specific capture antibody as the extraction phase. The analyte is bound to the antibody-coated segmented-nanoparticle and is detected with a second antibody tagged with a fluorescent dye, which may recognize a different epitope on the analyte. Similarly, an analyte bound to its receptor could be detected with an appropriate second antibody tagged with a fluorescent dye which recognizes an epitope on the receptor.

This process can be done in the same sample for as many proteins as there are both capture and detection antibodies. Several hundred pairs of antibodies are currently available. Thus, this process makes it possible to simultaneously interrogate a biological sample for the presence of all known proteins for which matched antibody pairs are available. Moreover, only one fluorophore needs to be used for the entire panel of assays run in the sample.

Since the potential number of flavors of segmented-nanoparticles far exceeds the number of available reagents, the same platform may also be able to detect post-translationally modified proteins, which are good candidates for new disease markers. Any protein can be subjected to co- and post-translational modifications. These modifications may have an influence on the charge, hydrophobicity, and conformation with respect to the “parent protein”, and can occur at different levels. Modifications such as acetylation, phosphorylation, methylation, hydroxylation, N- and O-glycosylation, can occur at the cellular level as well as in extracellular fluids.

To detect post-translational modification, polyclonal antibodies raised against a protein may be conjugated to a selected segmented-nanoparticle solid support. The polyclonal antibody will capture not only the protein against which it has been raised, but also protein isoforms (i.e., proteins that share similar epitopes but are modified at different sites). If the isoform is recognized by the detection antibody, it will be quantitated along with the “parent protein” (i.e. by the fluorescence immunoassay). If post-translational modification has affected the epitope that is recognized by the detection antibody, the isoform will not be quantitated by fluorescence. If both the capture antibody and the detection antibody are polyclonal antibodies, at least a great number of the modified proteins will be quantified. After the fluorescence measurement, the proteins may be captured by the segmented-nanoparticle extraction probes may be subjected to mass spectrometry analysis, either before or after elution.

Characterization of the proteins by SALDI-MS also identifies the different post-translation modifications. The SALDI-MS laser energy ruptures all non-covalent bonds allowing for detection of any molecule complexed on the extraction probes, including even the protein sandwiched by two antibodies. This highlights the importance of the ability to tag an assay with a specific segmented-nanoparticle extraction probe. The information encoded by the extraction probe will be associated with a specific protein having a specific molecular weight. When this probe is analyzed, instead of a full scan analysis, the mass spectrometer may be tuned to concentrate on a particular mass by using a technique called Single Ion Monitoring (SIM). SIM mode will allow faster acquisition of data and will increase the analytical sensitivity (up to 1000-fold enhancement in detecting an ion in SIM mode versus detecting this same ion in full scan mode). With the knowledge of the expected mass (and the sequence) of the analyte, the mass analyzer may be focused on a mass range allowing the detection of all the possible isoforms related to the parent protein. The monitoring range may be set to the molecular weight of the parent +/−500 Da, for example. Determination of the molecular weight of the isoform reveals immediately the modifications that the parent protein has undergone. Thus, the combination of segmented-nanoparticle solid support and polyclonal antibodies has the advantage of localizing the parent proteins as well as the corresponding isoforms on one flavor of segmented-nanoparticle extraction probe. Thus, the segmented-nanoparticles allow for a connection between the “parent protein” and the corresponding isoforms. This is not the case for 2-D gel electrophoresis where the post-translationally modified protein can appear in a different place of the gel if the charge has changed (following phosphorylation, for example). In short, 2-D gel requires a large amount of additional effort (such as sequence determination) for the identification of the modified protein because the connection between proteins of the same family is missing. This application of the invention is extended to any analyte and analyte isoform that are both capable of interacting with an extraction phase designed to extract the analyte.

The segmented-nanoparticle extraction probe platform also enables the investigation of protein-protein interactions by incorporating specific proteins in the extraction phase of the probes. This allows screening of biological samples for entities capable of interacting with those proteins (i.e., “molecular recognition”).

Segmented-nanoparticle extraction probe technology combined with fluorescence-based quantitation and mass spectrometer-based identification also allows investigation of specific protein-protein interactions. For instance, a biological sample may be interrogated for the presence of free analyte or for the presence of the free receptor for analyte by using a set of segmented-nanoparticles with antibodies for the analyte and receptor, respectively, and fluorescent detection antibody. Another set of nanorod extraction probes labeled with antibody directed against analyte can be used to quantify analyte bound to its receptor by using a detection antibody directed against the receptor. Similar assays can be set-up in which free auto-antibodies and auto-antibodies bound to analyte can be quantified using a fluorescent anti-Fc antibody, for example.

As described above, the segmented-nanoparticle extraction probes may be analyzed by a number of detection systems. Analysis by SALDI-TOF MS, for example, allows identification of the different isoforms present. However, even for cases where no detection antibodies are available, SALDI-TOF is still able to identify and characterize the different components. Quantitation by fluorescence will be missing, but identification by mass spectrometry of the captured analyte will still be possible. Alternatively a segmented-nanoparticle with an extraction phase comprising any protein can be used to pan the biological sample for the presence of a protein or any other entity having any affinity for said conjugated protein. The presence of the bound protein may be detected by mass spectrometric analysis.

The present invention also allows integration of various separation techniques with detection systems. As discussed above, in order to maximize the utility of available separation techniques, researchers have resorted to using two or more different separation methods to obtain separation. However, difficulties are frequently encountered in integrating the various separation techniques with each other and with the detection system. For example, it commonly proves difficult to maintain resolution upon transfer to the second dimension. Another major difficulty is that there is often a lack of compatibility between the mobile phases and the detection system. For example, salts and detergents in the eluant are incompatible with electrospray mass spectrometry. As another example, additional procedures must be taken to “clean-up” a sample before MALDI-MS analysis because the use of certain preservatives (e.g., chaotropes and solubilizing agents) suppress ionization.

Additionally, biological sources often contain a complex mixture of inorganic salts, buffers, chaotropes, preservatives, and other additives—often at concentrations higher than those of the molecules of interest—some of which are detrimental to MALDI MS. The segmented-nanoparticle (Nanobarcodes™ identification tags) approach used in the present invention is not only capable of generating as many extraction phases as the number of molecules present in a biological sample, it can also bind the molecules of interest to be analyzed by mass spectrometry. Accordingly, it provides an excellent mode of sample preparation prior to analysis. For example, segmented-nanoparticle extraction probes may be interrogated directly in MALDI/MS. Addtionally, because of their small size, the segmented-nanoparticle extraction probes may be directly introduced into ESI in which case the analytes absorbed by the extraction phase are released as molecular ions. Alternatively, the analytes may be introduced into MALDI/MS or ESI after they have been eluted from the segmented-nanoparticle extraction probe.

Although the above discussion has focused on the highly preferred segmented-nanoparticle extraction probes, one of skill in the art will recognize that other encodeable nanoparticles could be used according to the principles of the invention. For example, in one embodiment of the invention the particle solid supports are not comprised of segments, but are differentiable based on their size, shape or composition. Such an array of particles, which can be made up of any material, is comprised of at least 2, preferably at least 3, and most preferably at least 5 types of particles, wherein each type of particle is differentiable from each other type of particle. In the preferred embodiment, since the types of particles may be comprised of a single material and since different types of particles may be comprised of the same material as other types of particles in the assembly, differentiation between the types is based on the size or shape of the particle types. For example, an assembly of particles of the present invention may be comprised of 5 different types of gold rod-shaped nanoparticles. Although, each type of rod-shaped particle have roughly similar widths or diameters, the different types of particles may be differentiable based on their length. In another example, 7 types of spherical silver particles make up an assembly. The different types of particles are differentiable based on their relative size. In yet another example, 8 types of rod-shaped particles, all composed of the same polymeric material, make up an assembly; although each type of rod-shaped particles have the same length, they are differentiable based on their diameter and/or cross-sectional shape.

A further example of an array of nanoparticle solid supports within this embodiment of the invention is an assembly of particles, each type of which may have the same size and shape where the particle types are differentiable based on their composition or mass. For example, an array of particles of the present invention may be comprised of 5 different rod-shaped nanoparticles of the same size and shape. In this example, the different types of particles are differentiable based on the material from which they were made. Thus, one type of nanorod is made from gold, another from platinum, another from nickel, another from silver, and the remaining type from copper. Alternatively, each particle type may contain a different amount of a dye material, or a different percentage of magnetizable metal. In each case, a given particle type would be differentiable from the other particle types in the assembly or collection.

Of course, this embodiment of the invention includes arrays in which combinations of size, shape and composition are varied. The critical aspect of the array of particles of this embodiment is that the particle types are differentiable, by any means, from the other particle types in the assembly. The different types of particles may be associated with an extraction phase and the differentiable characteristics of the type of particles encode the nature of the extraction phase. By encoding the nature of the extraction phase, it is meant that the specific identifiable features of the nanoparticle can be attached selectively to a specific extraction phase, so that a key or log can be maintained wherein once the specific particle type has been identified, the nature of the associated extraction phase is known (or can be determined).

Sewmented-Nanoparticles as Solid Supports

Segmented-nanoparticles of the type which are highly preferred as solid supports in the present invention are described in detail in U.S. patent application Ser. No. 09/677,198, filed Oct. 2, 2000, entitled “Colloidal Rod Particles as Nanobar Codes,” incorporated herein in its entirety by reference.

Because bar coding is so widely-used in the macroscopic world, the concept has been translated to the molecular world in a variety of figurative manifestations. Thus, there are “bar codes” based on analysis of open reading frames, bar codes based on isotopic mass variations, bar codes based on strings of chemical or physical reporter beads, bar codes based on electrophoretic patterns of restriction-enzyme cleaved MRNA, bar-coded surfaces for repeatable imaging of biological molecules using scanning probe microscopies, and chromosomal bar codes (a.k.a. chromosome painting) produced by multi-chromophore fluorescence in situ hybridization. All these methods comprise ways to code biological information, but none offer the range of advantages of the bona fide bar codes transformed to the nanometer scale.

The highly preferred segmented-nanoparticles to be used as solid supports according to this embodiment of the invention are alternately referred to as segmented-nanoparticles, Nanobarcodes™ identification tags, nanorods, rods, and rod shaped particles. To the extent that any of these descriptions may be considered as limiting the scope of the invention, the label applied should be ignored. For example, although in certain embodiments of the invention the nanoparticle's composition contains informational content, this is not true for all embodiments of the invention. Likewise, although nanometer-sized particles fall within the scope of the invention, not all of the particles of the invention fall within such size range.

In highly preferred embodiments of the present invention, the nanoparticle solid supports are segmented-nanoparticles (Nanobarcodes™ identification tags) made by electrochemical deposition in an alumina or polycarbonate template, followed by template dissolution, and typically they are prepared by alternating electrochemical reduction of metal ions, though they may easily be prepared by other means, both with or without a template material. Typically, the segmented-nanoparticles have widths between 30 nm and 300 nanometers, though they can have widths of several microns. Likewise, while the lengths (i.e. the long dimension) of the materials are typically on the order of 1 to 15 microns, they can easily be prepared in lengths as long as 50 microns, and in lengths as short as 10 nanometers. In some embodiments, the segmented-nanoparticles comprise two or more different materials alternated along the length, although in principle as many as dozens of different materials could be used. Likewise, the segments could consist of non-metallic material, including but not limited to polymers, oxides, sulfides, semiconductors, insulators, plastics, and even thin (i.e., monolayer) films of organic or inorganic species.

When the segmented-nanoparticles are made by electrochemical deposition, the length of the segments can be adjusted by controlling the amount of current passed in each electroplating step; as a result, the nanorod resembles a “bar code” on the nanometer scale, with each segment length (and identity) programmable in advance. The same result could be achieved using another method of manufacture in which the length or other attribute of the segments can be controlled. While the diameter of the nanorods and the segment lengths are typically of nanometer dimensions, the overall length is such that in preferred embodiments it can be visualized directly in an optical microscope, exploiting the differential reflectivity of the metal components.

The synthesis and characterization of multiple segmented particles is described in Martin et al., Adv. Materials, 11, 1021-25 (1999). The article is incorporated herein by reference in its entirety. See also, U.S. patent application Ser. No. 09/677,203, filed Oct. 2, 2000, entitled, “Method of Manufacture of Colloidal Rod Particles as Nanobar Codes,” and U.S. patent application Ser. No. 09/676,890, filed Oct. 2, 2000, entitled, “Methods of Imaging Colloidal Rod Particles as Nanobar Codes,” both incorporated herein in their entirety by reference.

In certain preferred embodiments, the segmented-nanoparticle solid supports of the present invention are defined in part by their size and by the existence of at least 2 segments. The length of the nanoparticles can be from 10 nm up to 50 μm. In preferred embodiments the nanoparticle is 500 nm to 30 μm in length. In the most preferred embodiments, the length of the nanoparticles of this invention is 1 to 15 μm. The width, or diameter, of the particles of the invention is within the range of 5 nm to 50 μm. In preferred embodiments the width is 10 nmn to 1 μm, and in the most preferred embodiments the width or cross-sectional dimension is 30 nm to 500 nm.

A segment represents a region of the particle that is distinguishable, by any means, from adjacent regions of the nanoparticle. Segments of the nanoparticle typically bisect the length of the nanoparticle to form regions that have the same cross-section (generally) and width as the whole nanoparticle, while representing a portion of the length of the whole nanoparticle. In preferred embodiments of the invention, a segment is composed of different materials from its adjacent segments. However, not every segment needs to be distinguishable from all other segments of the nanoparticle. For example, a nanoparticle could be composed of 2 types of segments, e.g., gold and platinum, while having 10 or even 20 different segments, simply by alternating segments of gold and platinum. A segmented-nanoparticle used in the present invention contains at least two segments, and as many as 50. The nanoparticles preferably have from 2 to 30 segments and most preferably from 3 to 20 segments. The nanoparticles may have from 2 to 10 different types of segments, preferably 2 to 5 different types of segments.

A segment of the nanoparticle is defined by its being distinguishable from adjacent segments of the nanoparticle. The ability to distinguish between segments encompasses distinguishing by any physical or chemical means of interrogation, including but not limited to electromagnetic, magnetic, optical, spectrometric, spectroscopic and mechanical. In certain preferred embodiments of the invention, the method of interrogating between segments is optical (e.g., reflectivity).

Adjacent segments may even be composed of the same material, as long as the segments are distinguishable by some means. For example, different phases of the same elemental material, or enantiomers of organic polymer materials can make up adjacent segments. In addition, a rod comprised of a single material could be considered to fall within the scope of the invention if segments could be distinguished from others, for example, by functionalization on the surface, or having varying diameters. Also nanoparticles comprising organic polymer materials could have segments defined by the inclusion of dyes that would change the relative optical properties of the segments.

The composition of the segmented-nanoparticle solid supports of the present invention is best defined by describing the compositions of the segments that make up the nanoparticles. A nanoparticle may contain segments with extremely different compositions. For example, a single nanoparticle could be comprised of one segment that is a metal, and a segment that is an organic polymer material.

The segments may be comprised of any material. In preferred embodiments of the present invention, the segments comprise a metal (e.g., silver, gold, copper, nickel, palladium, platinum, cobalt, rhodium, iridium); any metal chalcognide; a metal oxide (e.g., cupric oxide, titanium dioxide); a metal sulfide; a metal selenide; a metal telluride; a metal alloy; a metal nitride; a metal phosphide; a metal antimonide; a semiconductor; a semi-metal. A segment may also be comprised of an organic mono- or bilayer such as a molecular film. For example, monolayers of organic molecules or self assembled, controlled layers of molecules can be associated with a variety of metal surfaces.

A segment may be comprised of any organic compound or material, or inorganic compound or material or organic polymeric materials, including the large body of mono and copolymers known to those skilled in the art. Biological polymers, such as peptides, oligonucleotides and carbohydrides may also be the major components of a segment. Segments may be comprised of particulate materials, e.g., metals, metal oxide or organic particulate materials; or composite materials, e.g., metal in polyacrylamide, dye in polymeric material, porous metals. The segments of the nanoparticles used in the present invention may be comprised of polymeric materials, crystalline or non-crystalline materials, amorphous materials or glasses.

Segments may be defined by notches on the surface of the nanoparticle, or by the presence of holes or perforations into the particle. In embodiments of the invention where the nanoparticle is coated, for example with a polymer or glass, the segment may consist of a void between other materials.

The length of each segment may be from 10 nm to 50 μm. In preferred embodiments the length of each segment is 50 nm to 20 μm. The interface between segments, in certain embodiments, need not be perpendicular to the length of the nanoparticle or a smooth line of transition. In addition, in certain embodiments the composition of one segment may be blended into the composition of the adjacent segment. For example, between segments of gold and platinum, there may be a 5 to 50 nm region that is comprised of both gold and platinum. This type of transition is acceptable so long as the segments are distinguishable. For any given nanoparticle the segments may be of any length relative to the length of the segments of the rest of the nanoparticle.

As described above, the segmented-nanoparticle solid supports can have any cross-sectional shape. In preferred embodiments, the nanoparticles are generally straight along the lengthwise axis. However, in certain embodiments the nanoparticles may be curved or helical. The ends of the nanoparticles of the present invention may be flat, convex or concave. In addition, the ends may be spiked or pencil tipped. Sharp-tipped embodiments of the invention may be preferred when the nanoparticles are used in Raman spectroscopy applications or others in which energy field effects are important. The ends of any given nanoparticle may be the same or different.

A key property of certain embodiments of the nanoparticle solid supports of the present invention is that when the particles are segmented, differences in the reflectivities of the component metals can be visualized by optical microscopy. Thus, in a segmented Au/Pt/Au rod of 200 nm in diameter and 4 to 5 microns in overall length, the segments are easily visualized in a conventional optical microscope, with the Au segments having a gold lustre, and the Pt segments having a more whitish, bright lustre. Another key property of the materials is that the length of the segments, when they are prepared by alternating electrochemical reduction of two or more metal ions may be controlled (and defined) by (a) the composition of the solution and (b) the number of Coulombs of charge that are passed in each step of an electrochemical reduction. Thus, the widths and the number of the segments can be varied at will.

The ability to identify segmented-nanoparticles via their reflectivity and the ability to modify their surfaces with biomolecules allows the nanorods to be used as solid supports and optical tags simultaneously.

What distinguishes segmented-nanoparticles from other types of optical tags, or indeed from any type of tag ever applied to a molecular system (including isotopic tags, radioactive tags, molecular tags for combinatorial beads, fluorescence-based tags, Raman-based tags, electrochemical tags, and other tags known to those of skill in the art,) is the essentially unlimited variability. With the ability to use 7 or more different metals, 20 or more different segments, and 4 or more different segment lengths, and with 3 or more different rod widths, there are essentially an arbitrarily large number of different segmented-nanoparticles that can be prepared. Even with just two types of metals and just 10 segments, with just one segment length, and with just one rod width, over a thousand different types (“flavors”) of segmented-nanoparticle can be prepared.

The segmented-nanoparticle solid supports of the present invention can be read using existing instrumentation, e.g., chemical force microscopy, optical readers, etc. However, instrumentation and software specifically designed to identify segmented-nanoparticles are also contemplated within the scope of this invention. Specifically included within the scope of the invention are modified Micro Volume Laser Scanning Cytometry (MLSC) apparatus and modified flow cytometer apparatus that can be used to image or read segmented-nanoparticles.

It should be noted, however, that a variety of detection mechanisms for reading the nanoparticle solid support can be used, including but not limited to optical detection mechanisms (absorbance, fluorescence, Raman, hyperRaman, Rayleigh scattering, hyperRayleigh scattering, CARS, sum frequency generation, degenerate four wave mixing, forward light scattering, back scattering, or angular light scattering), scanning probe techniques (near field scanning optical microscopy, AFM, STM, chemical force or lateral force microscopy, and other variations), electron beam techniques (TEM, SEM, FE-SEM), electrical, mechanical, and magnetic detection mechanisms (including SQUID). Significantly, the stripes of segmented-nanoparticles are spatially resolved. This property differentiates them from, for example, color coded microspheres. The segmented-nanoparticles of the invention can thus be interrogated in ways that exploit the spatial resolution of their properties (e.g., differential reflectivity of specific stripes), as well as methods that measure bulk properties.

The sensitivity of hyperRaleigh scattering may make it a particularly useful interrogation technique for reading the nanoparticle solid supports of the present invention. For example, see, Johnson et al, The Spectrum, 13, 1-8 (2000), incorporated herein in its entirety by reference.

Needles as Solid Supports

Another means of achieving multiplexing of solid phase extraction on the micro or nanoscale is to employ arrays of extraction probes comprised of fibers (“needles”) as the solid support. Indeed, configured as arrays, the fibers can be coated with extraction phase and exposed to the sample in a multiplexed fashion that lends itself to automation. Furthermore, presentation to a suitable analytic instrumentation is also facilitated. When the needle solid supports are configured in arrays, solid phase microextraction may occur simultaneously.

For example, the present invention contemplates a set of extraction probe needles being simultaneously inserted into an apparatus that provides a variable delay into a single analytic instrument for the analysis of analytes. This variable delay allows for sequential analysis; in other words, it is not necessary to have “N” analytical instruments for “N” needles; N extraction probe needles can be analyzed with a single instrument, if the analyses can be time-staggered. This approach is utilized for LC interfaces to mass spectrometry. The same concept would also work for introduction into LC, CE or GC analytic instrumentation.

The present invention includes an array of solid needles or rods that are coated with an extraction phase to provide for separation of analytes by differential affinity with respect to the extraction phase. The array may be comprised of solid supports that are needles, pinheads, rods or any other suitable solid objects. Extraction phases include, but are not limited to, C18, C8, hydroxyapatite, anion or cation exchange resins or material used for affinity chromatography, inorganic materials, metal alloys, oxides, glasses, ceramics, zeolites, polyelectrolyte multilayers, etc., or combinations thereof.

An example protocol of the method of the present invention is as follows: The array of extraction probes is washed with a suitable reagent/solvent to disengage all bound molecules. The array is equilibrated in an appropriate solution to assure correct conditions for binding in the next step. The array is soaked with mild stirring in a mixture of analytes containing one or more components that may bind to the extraction phase. The array is lifted from the mixture and introduce into a wash solution such that unbound analytes may be washed away. This step may be repeated several times. For extraction of bound analytes, the array is introduced into a solvent/solution that allows disengagement of the bound analytes into the extraction solvent. The extraction solvent then contains the analytes preferentially bound to the arrayed separation phase.

Experiments using the arrays can be designed in a number of ways. The experiments can use the same extraction phase in the array with different mixture samples. Alternatively, the extraction phase can be used to isolate different analytes from the same sample by employing different extraction phases on each rod/needle.

For example, DNA may be separated from salts using C18 resin as the extraction phase as follows. First, a 96-well array of C18 coated needles is washed three times with Acetonitrile and then equilibrated in 50 mM triethylammonium acetate buffer, pH 6.5. Next, the array is soaked in a mixture of DNA and salts, allowing the DNA to bind to the C18 resin. Then the array is removed from the mixture and washed three times in the equilibration buffer (TEAA) to remove the unbound salts. Finally, the bound DNA is extracted into 50% Acetonitrile.

To further achieve the advantages of combinatorial solid phase extraction techniques of the present invention, it is important to have as large a number of extraction phases available for extraction as possible. This can be accomplished by increasing the number of arrayed extraction probes that are contacted with the sample. Because up to thousands of parallel experiments are contemplated for the preferred embodiments of the present invention, it would be impractical to use fibers of the size conventionally used in solid phase extraction techniques. Thus the arrayed extraction probes of the present invention can be reduced in size so that they are on the micron or submicron (i.e., nanometer) scale.

As conventionally practiced, the needle-shaped fibers used in SPME are injected into HPLC, CE, or GC instrumentation using traditional injection ports. However, the use of microfluidic devices enables the use of miniaturized collection means as an alternate detection/analysis embodiment. Current microfluidic devices typically may use anywhere from nanoliters to femtoliters of solvent. Such devices may be coupled to nanofiber extraction probes for analyte desorption and delivery. For such embodiments, conventionally sized needle-shaped fibers would deliver far too much material to be practical.

Microfluidic devices may be replicated precisely and in large numbers at low cost. Because microfluidic-based separations occur in devices that have small physical dimensions, they may be stacked together, or otherwise concatenated. In fact, 1000 or more microfluidic devices may be clustered, each capable of extracting the contents of an individual nanofiber extraction probe. In contrast, it would be impractical to carry out 1000 sequential separations using one CE instrument, or even 100 with 10 CE instruments.

As technology advances and microfluidic devices become smaller, and it will be useful for the extraction probe needles to become smaller. Thus, another aspect of the present invention is the use of needle extraction probe arrays that cannot be prepared by conventional means owing to their small size. For example, needles with a diameter of less than 1 micron (μm) may be prepared photolithographically. Using deep-ultraviolet or projection lithography, features as small as 100 nm are easily attainable. Through the use of this or equivalent technology, needle extraction probe arrays may be synthesized that are of nanometer dimensions. Likewise, a plate of wells in which the well-well spacing is identical to spacing between needles can also be fabricated. As described earlier for larger scale extraction probes, this would allow each needle to be coated with a different extraction phase, by filling each well with a different chemistry. This may be accomplished, for example, by putting into each well (of a multi-well plate) a single bead from a split pool synthesis. The compound on the bead may be known or unknown. The compound is then released from the bead by one of a number of means known in the art, and then reacted with a functional group on the surface of the solid support array of needles. In this way, each needle would harbor a different ligand. This needle extraction probe array could then be introduced into wells of another multi-well plate containing a sample (e.g., wherein each well contains a divided aliquot of the sample). The receptors in the sample with affinity (medium or high) for a particular needle-associated ligand will be extracted by the corresponding extraction phase. Alternatively, in another embodiment, the needles from the array could be reacted with the same functional group so that each needle would harbor the same ligand. This needle extraction probe array could then be introduced into wells of another multi-well plate in which each well contains a different sample.

Another example of the application of ultra-miniature extraction probe needle arrays involves the use of lower affinity materials. As described below, libraries of 10,000 polymers may be generated using the technique of combinatorial materials synthesis wherein each of the polymers has a different structure and a different affinity for molecules or classes of molecules. Indeed, the synthesis could be designed to yield highly diverse properties for these materials. Alternatively, the combinatorially generated polymers could be selected (perhaps based on empirical tests) for their diverse properties. Subsequent immobilization of these combinatorially obtained polymers onto ultraminiature solid support needle arrays would enable a set of parallel nano-extraction experiments that broadly sampled the molecular structure space contained in a complex sample mixture. These experiments will involve the use of “nano-needles,” because the analyte volume will be split up into many thousand identical aliquots. For example, with 10,000 needles, and 10 microliters of total sample, each well would contain one nanoliter of solution. This translates to a cube of 0.01 cm on each side. hi other words, the extraction probe needle would have to be no more than 10 microns long, and about 1 micron wide.

Needle extraction probe arrays do not require the use of combinatorially-derived libraries to furnish useful extraction phase chemistries. To the contrary, such needle arrays can exploit any collection or combination of extraction phase, from commercially available chromatographic resins to monoclonal or polyclonal antibodies to oxide materials.

The present invention allows several methods for solid phase micro- or nanoextraction of analytes in parallel. For example, chromatographic media could be placed as a micro column within a pipette tip called a Zip-Tip (Millipore) or, alternatively, coated on the inner surface of a pipette tip such as Supro Tip and Pro Tip (Amika Corporation/Harvard Apparatus). These are hollow objects with chromatographic media coated or as a plug in the hollow structure. A multichannel pipettor would allow parallel processing using these tips.

In addition to coated solid objects, the arrayed objects may be porous and comprised of packed chromatographic media in the pores. Indeed, in certain of such embodiments, each needle may also be attachable to a charge source that allows a range of voltages to be applied to any given needle in the array. In this way, the needle can differentially extract components of the sample based on charge. These arrayed, immobilized chromatographic media can be used for isolation from a mixture of analytes that may preferentially bind to the selected media.

Beads as Solid Supports

Another aspect of this invention includes using beads as the solid support for producing a bead-based extraction probes for solid phase extraction. The bead extraction probes are contacted with the sample to be analyzed and subsequently collected and separated (e.g., by centrifugation). The bound analyte can then be analyzed. If analyzed by mass spectrometry, the analyte may be first eluted, or analysis may take place directly without elution.

In a simple embodiment of this approach, a collection of beads with the same extraction phase on each bead is used. This solution-based approach has several advantages over simple one-channel SPME as it is currently practiced. One important advantage is that equilibration time is shortened using a collection of beads. Because both the extraction phase and the analyte are mobile, encounters between them occur more frequently and the capture of analyte molecules is more rapid. This is of particular importance for applications in which the sample volume is large (e.g., in environmental samples), or where analyte is present in sufficiently low concentration to warrant concentration.

Another important advantage to using bead-based extraction probes is the increase in surface area available with small, three-dimensional particles compared to that of a fiber or needle. To illustrate: The surface area of a 5 mm cylinder of 100 micron diameter is approximately 1.57 mm². When the same volume is made up from a collection of 6,000 beads of 5 micron diameter, the total surface area is 47.1 mm², a factor of 30 greater. The increased surface area in the bead-based approach provides another factor that will lead to a more rapid equilibration. Equally important, an increase in the surface area means that the capacity of the extraction phase will increase, because a given volume is more accessible in the form of spherical particles, where 3-D diffusion is allowed, than in a monolithic solid.

Another advantage to using a bead-based extraction probe is the ability to access samples that would otherwise be difficult to obtain. For example, if one wanted to carry out solid phase micro- or nanoextraction on whole blood in circulation, one could use beads smaller than the diameters of capillaries. Alternatively, larger solid support beads could also be used if the animal were to be sacrificed at the end of the experiment, or if the bead extraction probes could be localized within certain body compartments (either natural or artificially created). To collect these beads, one could make them magnetic, thus allowing them to be readily removed from an organism after a certain time. Beads can be made magnetic by incorporation of magnetic material on the interior, exterior, or both. Magnetic retrieval allows the bead extraction probes to be isolated with minimal sample perturbation, for example, in applications where centrifugation is disfavored (i.e., in whole blood). Of course, numerous alternative methods of bead retrieval are available, including without limitation, centrifugation, gravity-based particle settling (in a non-gradient containing or gradient containing column, or even in solution), optical methods (e.g., optical trapping), and bead based flow/sorting methods (e.g., using cytometry and fluorescence-activated cell sorting (FACS)). In addition, bead-based extraction probes may be collected using microfluidic devices by one of a number of different methods, including isoelectric focusing, dielectrophoresis, acoustic focusing, among a number of others. In addition to being collected, particles can also be sorted by dielectrophoretic trapping (see, e.g., Proc. SPIE, 4177, 164-173 (2000)) or by a number of analogous methods.

Bead-based solid phase extraction separations may be conducted in a number of ways. In one embodiment, numerous beads are used, each with the same extraction phase. More powerful is the embodiment where subsets of beads have different extraction phases, such as those described above with respect to segmented-nanoparticles and needle arrays. For example, one could use eight different kinds of bead extraction probes, with high affinities for (i) carbohydrates, (ii) acids, (iii) bases, (iv) hydrophobic compounds, (v) hydrophilic compounds, (vi) aromatic compounds, (vii) metal cations, and (viii) inorganic anions. This list could be expanded, of course, to hundreds or thousands of different extraction phases as needed, encompassing specific capture agents such as oligonucelotides and/or antibodies as well as ligands for particular receptors, cofactors for proteins, and so forth.

The different extraction phases that can be used on subsets of beads can be “low affinity” as well, including the standard extraction phases commonly used in SPME, and designed or selected low affinity extraction phases. Furthermore, combinations of high affinity and low affinity bead extraction probes could be instrumental in sampling a large fraction of a complex sample.

While bead-based extraction probes are typically understood to be spherical, the present invention is not so limited. Beads, particles, or objects of any shape or size, can be used and are contemplated by the present invention as solid supports, so long as they can be made to be coated with, attached to, or associated with an extraction phase. Thus, collections of spherical nanoparticles of any dimension could be used, as could cylindrically-shaped particles of any size. Indeed, a collection of particles of different sizes, shapes and compositions could be used as well. For example, one could use 10 nm diameter metal nanoparticles with a first extraction phase, 50 nm diameter latex particles with second extraction phase, 3 micron cylindrical oxide particles with a third extraction phase, and so on. In short, any single size, shape or composition of particles, or any combination of sizes, shapes, and compositions can be used. By analogy, the same is true for needle extraction probe arrays on the submicron scale.

It is not necessary that the beads used for solid phase extraction be encoded; that is, contain information that can be used to differentiate or identify the beads or particles. However, as described above with respect to nanoparticles, significant benefits are achieved when such encoding is used.

Combinatorial Solid Phase Extraction

One of the embodiments of the present invention is referred to herein as combinatorial solid phase extraction. That is, solid phase extraction in which a large number of empirically-chosen microextractions are carried out in parallel (or serially in rapid succession). This number can be as few as 3, and as great as 10,000,000. Useful embodiments include numbers between 4 and 100,000, and also between 10 and 1,000.

Combinatorial approaches have met with success in several fields. For example, combinatorial synthesis of possible drug candidates has found acceptance in medicinal chemistry. Likewise, combinatorial discovery of materials has become popular via approaches that lead to thousands or even millions of unique compositions of polymers, oxides, ceramics, etc. These advantages can be brought to the generation of materials to be used as extraction phases.

Thus, a number of nanoparticles can be coated, each with different extraction phases, including, for example, an antibody or other solid phases resulting in a library of unique nanoparticle extraction probes. Multiple nanoparticle extraction probes can interact with the sample at the same time. Differential analysis is possible because it is known (or can be determined) what extracting phase was associated with a given nanoparticle.

The coating of the solid support may be accomplished using an automated approach. The components to be coated on the solid support can be obtained by combinatorial methods known in the art. Indeed, the synthesis of the material or materials making up the extraction phase on the nanoparticle lends itself to combinatorial approaches where the automated, parallel synthesis of thousands, or even hundreds of thousands, of chemical variations is possible. Likewise, combinatorially derived extraction phases could be applied to arrays of fibers (or “needles”) which are then exposed to the sample in a multiplexed fashion. The application of the extraction phases as well as the contacting the array with the sample and presentation to a suitable analytic instrumentation could be readily automated. The nature of the associated extraction phase can be readily identified by the spatial address of the fiber.

The resulting diversity increases the resolution that can be obtained in the analysis of the biological sample. The greater number of extraction phases means that there is a greater chance that any given analyte will interact with an extraction probe. The large number of differentiable nanoparticles possible allows a diverse population of probes. Thus, the present invention allows for the creation of a vast library of nanoparticle extraction probes, with varying affinities for different molecules. When this diverse set of probes is added to a biological sample, incubated, washed, and analyzed by, for example, SALDI-MS, each nanoparticle probe will represent a particular extraction phase. As a whole, the ensemble will provide a fingerprint of the sample.

Such combinatorial solid phase extraction is distinct from multiplexed solid phase extraction. Multiplexed solid phase extraction refers to a number of measurements that are carried out in parallel (or serially in rapid succession). Combinatorial solid phase extraction, in contrast, refers to an empirical approach to synthesis that emphasizes generation of a large number of random or semi-random structures in order to find one or more with desired properties. Often, combinatorial syntheses are multiplexed in the sense that they are often carried out simultaneously in the same sample. For example, immunoassays in which multiple immunoassays are carried out simultaneously in the same sample volume are multiplexed, but are not combinatorial because they target a well-defined set of molecules. Similarly, when the species attached to the array surface in “gene chips” or oligonucleotide arrays are selected for the purpose of quantitation of complementary sequences, they may be multiplexed, but cannot be considered combinatorial.

Combinatorial solid phase extraction employs a large variety of extraction extraction phases with the goal of extracting as many species as possible from a complex mixture. The extraction phases contemplated by the present invention need not be limited to the type traditionally used for SPME needle experiments. Indeed, the extraction phases can run the gamut from monoclonal antibodies and oligonucleotides (that have high affinity for few species) to those used in chromatography or traditional SPME (i.e., with low affinity for a large number of species).

Examples of extraction phases that can be used in the combinatorial methods of the present invention include, but are not limited to, polymers, block copolymers, self-assembled monolayers and derivatives thereof, molecularly-imprinted polymers, hyperbranched polymers, dendrimers, polyelectrolytes, gels, glasses, oxides, ceramics, semiconductors, amorphous materials, nucleic acids, oligonucleotides, carbohydrates, polysaccharides, peptides, proteins, lipids, and other biological molecules. Additional examples include all known stationary phases that have been used in paper, thin-layer, liquid and gas chromatography. Additional examples also include individual members of combinatorial libraries or multiple members thereof. For example, solid phase extraction could be carried out with an extraction phase comprising latex modified with particular organic compounds that would exhibit a variable range of affinities for analytes in a sample. This could be replicated for numerous compounds from a library on numerous solid supports, each with a slightly different affinity.

Those skilled in the art will recognize that the extraction phase of a combinatorial solid phase extraction experiment can encompass virtually all known chemical structures, whether molecular or non-molecular (e.g., supramolecular, solid-state, etc.). Moreover, those skilled in the art will recognize that there are an arbitrarily large number of possible combinations of these solid phase extraction phases.

With the numbers of possible segmented-nanoparticles being enormous, combinatorial chemistry allows creation of an equally large number of compounds for inclusion in the extraction phase. Thus, it becomes important to be able to attach these compounds to (or otherwise associate these compounds with) segmented-nanoparticles (Nanobarcodes™ identification tags) in order to create combinatorially designed surfaces for analyte capture. Several methods are known in the art that could accomplish this attachment. They include: Self-assembled monolayers, monolayers that are not self-assembled, partial layers, deposited film and materials (including from gas phase and/or solution phase), multilayers, grown materials (i.e., deposited materials that are chemically transformed), etc. It should further be clear that the extraction phase could result from tranformation of the material intrinsic to the segmented-nanobar. In other words, the outermost portion an Al stripe can easily be converted to Al₂O₃, and this can be used as an extraction phase. Likewise, Si can converted to SiO₂, W to WO₃, and so forth. Indeed, an entire segment could be used as an extraction phase. For example, if one of the segments in a segmented-nanoparticle were porous glass, the entire segment itself could be used as an extraction phase. Moreover extraction phases could be prepared by addition of materials to the segments themselves. For example, a SiO₂ segment, upon treatment with a chiral reagent, could serve as an effective extraction phase for certain classes of chiral compounds. Finally, it should be clear to those skilled in the art that combinations of extraction phases on nanoparticles are both feasible, owing to the differentiable chemistry of non-identical segments, and desirable, insofar as a combination of extractions from various portions of an individual particle might comprise an improved separation relative to a single extraction from a single particle, or from a combination of single extractions from multiple particles. By the same token, it should be recognized that different amounts of the same extraction phase on different particles comprise distinct extraction phases, to the extent that they will bind different amounts of materials.

One means to generate these extraction phases is to use self-assembled monolayers (SAMs) terminated with reactive functional groups. These SAMs may be derivatized with libraries of reagents to give segmented-nanoparticle extraction probes with extraordinary variety in surface chemistry.

There are alternatives to using SAMs. For example, nanoparticles could be coated with polymers; the polymers could be synthetic organic polymers. Each polymer coat serving as an extraction phase can have selected properties. Monomers may be attached to the nanoparticles and the polymerization reaction conducted directly on the surface of the nanoparticle. (E.g., Mirkin, WO 99-U/S28387, “Preparation of Nanoparticles with Polymer Shells for Use in Assays.”).

Useful polymers may be inorganic such as an amorphous silica (i.e., glass) coat that may be polymerized on top of a thioalkyl silane SAM. Resulting amorphous silica can possess chemically active functional groups. Polymers could be directly adsorbed to the nanoparticle surface. This polymer layer would be stabilized through multipoint attachment (non-covalent). Examples include polylysines, aminodextrans, or selected proteins.

Each extraction phase could be uniquely designed to capture only one class of molecules. Such classes may include large molecules, such as proteins. In addition, combinatorially prepared extraction phases could be empirically tested to determine which molecules present in a sample were captured, and to what extent. If the desired criteria can be specified, such screening could be automated to take place in a high-throughput manner to determine the appropriate extraction phases that meet the desired criteria. For example, it may be that the presence of a certain metabolite is suspected of being a significant marker for a disease. Thousands of extraction probes could be prepared by coupling encoded particles (e.g., segmented-nanoparticles) with combinatorially prepared extraction phases, the code specifying the extraction phase or the method for its preparation. These extraction probes could then be screend against a sample known to contain the metabolite. The extraction probe, or set of extraction probes, found to best extract the metabolite (e.g., as assayed by mass spectrometry) could be determined from the encoded information and used subsequently to assay unknown samples. This same empirical method could be used to arrive at a set of extraction probes that produces a meaningful fingerprint of a sample. This would decrease the cost and time required to obtain useful information, because only the minimal number of probes that completely describe the sample would have to be synthesized and analyzed. Thus, rather than contact the sample with arbitrarily large number of unique extraction probes, a limited set (e.g., <50) could be used that have been found to pan the sample. The desirable set of extraction probes may be different for different samples. Thus, the set of extraction probes desirable for panning urine of diabetic patients would likely be different from the set of extraction probes desirable for panning synovial fluid from an arthritic patient.

Self-assembled monolayers formed from w-carboxy substituted alkanethiols on the surface of gold have been used as model surfaces to study the interactions of proteins with surfaces. (Mrksich et al., JACS, 117, 12009 (1995)). Derivatization of such nanoparticles may be achieved by various chemical means. One way involves “capping” with water soluble mercapto derivatives, typically mercapto carboxylic acid or amines. The carboxyl or amines are subsequently used to covalently label proteins, peptides or nucleic acids to give biomolecular conjugates of these particles that can be used in biological assays. This is discussed in, for example, Spinke et al., Langmuir, 9, 1821 (1993); Willner et al., J. Am. Chem. Soc., 114, 10965 (1992); and Mrksich et al., J. Am. Chem. Soc., 117, 12009 (1995).

Mixed SAMs formed from hydrophobic (alkyl, phenyl) and hydrophilic (hydroxyl, oligo ethylene glycol), positively charged (quaternary ammonium) and negatively charged (carboxylate, phosphate, sulphonate) species can be used in extraction phases to recognize and bind various molecules. Mixed SAMs have been used to study the adsorption of fibrinogen, lysozyme, pyruvate kinase, RNAse and carbonic anhydrase. (See, Lahiri et al., Anal. Chem., 71, 777 (1999); Prime et al., Science, 252, 1164 (1991)).

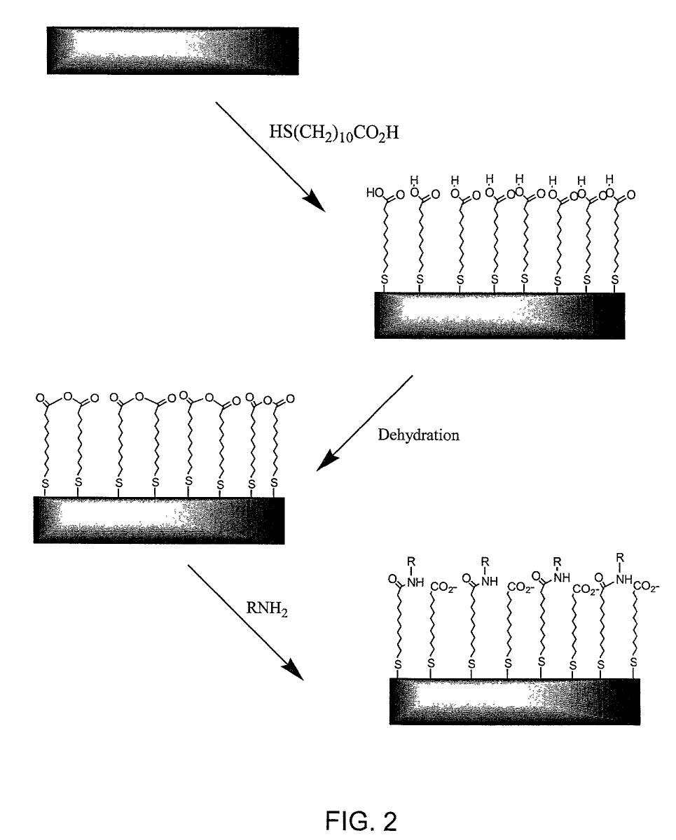

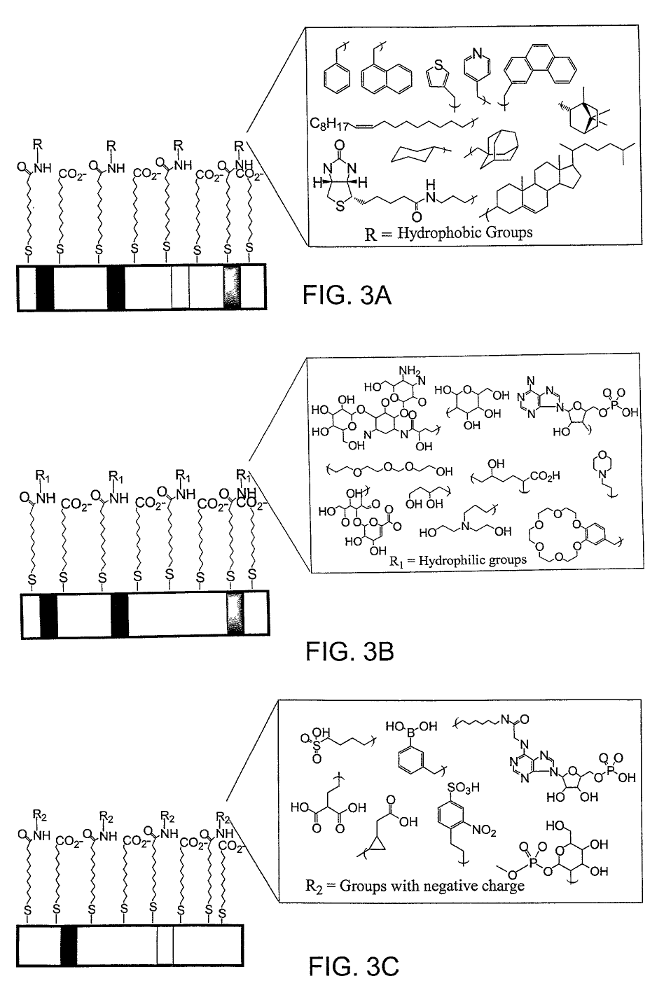

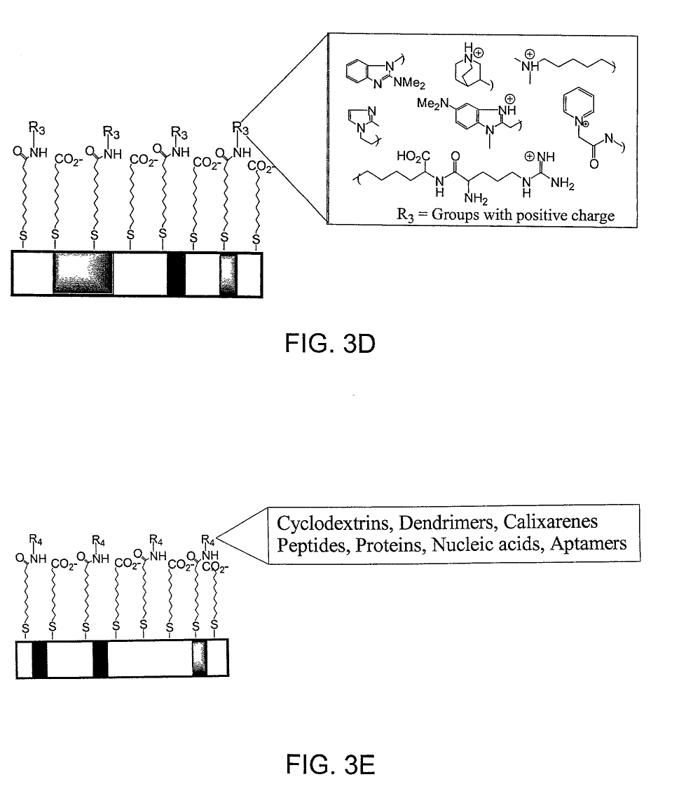

According to the present invention, nanorod solid supports are prepared that possess SAMs that terminate with carboxyl functionality. This is achieved by reacting the nanoparticles with ω-carboxy alkanethiols. The carboxyl functionality is then activated to an anhydride for further reaction with a wide variety of amines with diverse functional groups. This is illustrated in FIG. 2. FIGS. 3A-3E illustrate some of the potential classes of extraction phases and specific functional groups that can be generated by this method.

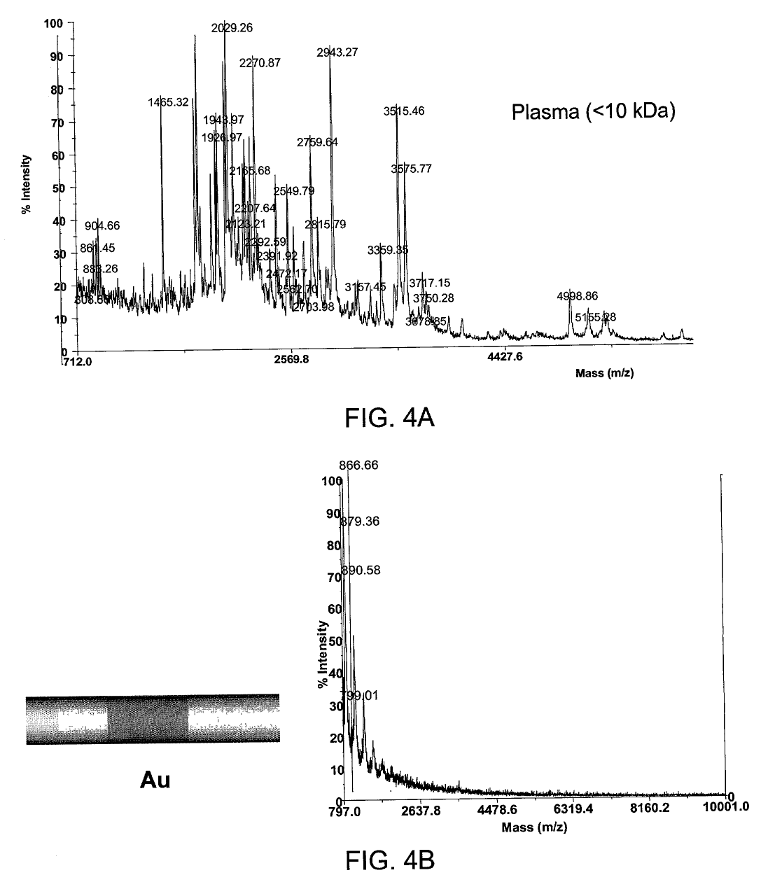

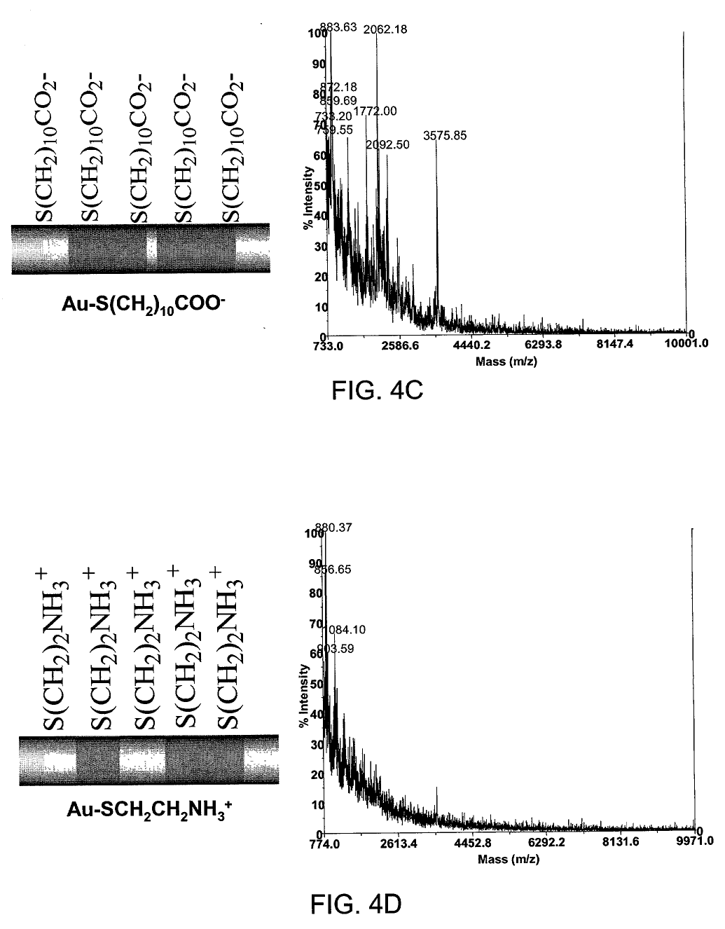

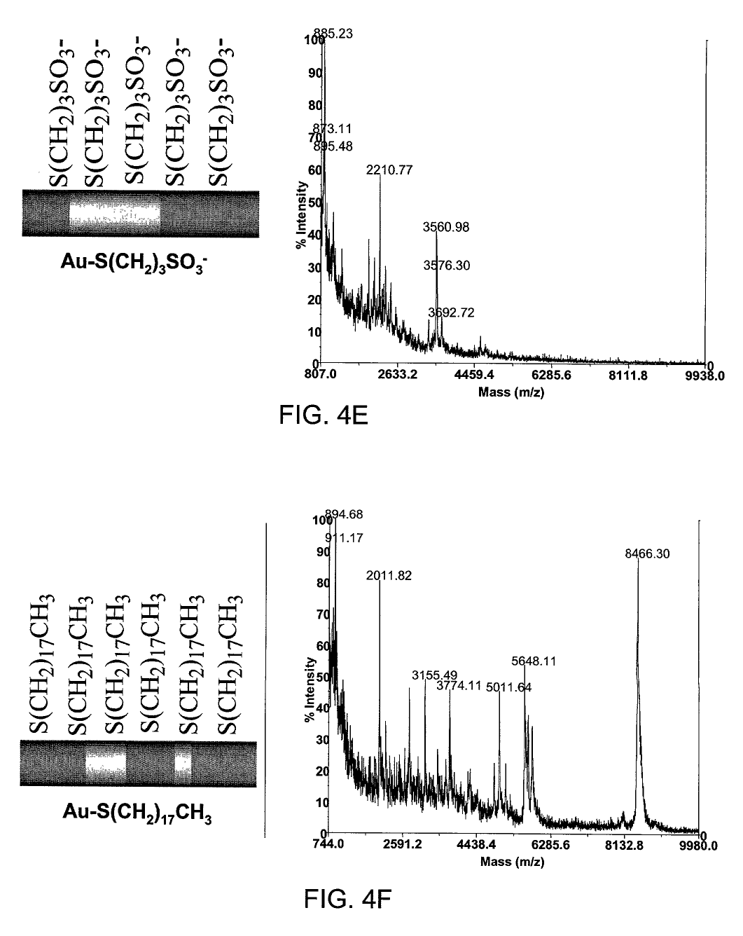

Encoded nanoparticle solid supports with SAM extraction phases have been prepared and used to perform SPNE from a low-molecular weight (<10 kDa) human plasma fraction. FIG. 4A shows the mass spectrum of the plasma sample before analyte extraction. The five different derivatized nanoparticles used are illustrated in FIGS. 4B-4F, along with MALDI mass spectra obtained from the separated nanoparticles after extraction. The extraction phases illustrated are bare gold, carboxyl-terminated, amine-terminated, sulfonate-terminated, and alkyl surfaces. As shown, each type of nanoparticle is encoded with a different striped pattern, allowing the particles to be contacted with the sample simultaneously and then separated based on the code. The different mass spectra illustrate that the different particles are indeed extracting different analytes from the complex biological sample. For example, the sulfonate and carboxyl groups extract positively charged analytes with different affinities. The amine groups extract negatively charged analytes, while the alkyl groups extract hydrophobic analytes. Note that the bare gold particles do not appear to extract many species, indicating that non-specific interactions are not particularly effective in performing the extraction.

Another class of derivatives that would provide amine reactive functionality as well as prevent non-specific interactions with proteins is dextran lactones. These can be prepared from carboxymethyl dextran.

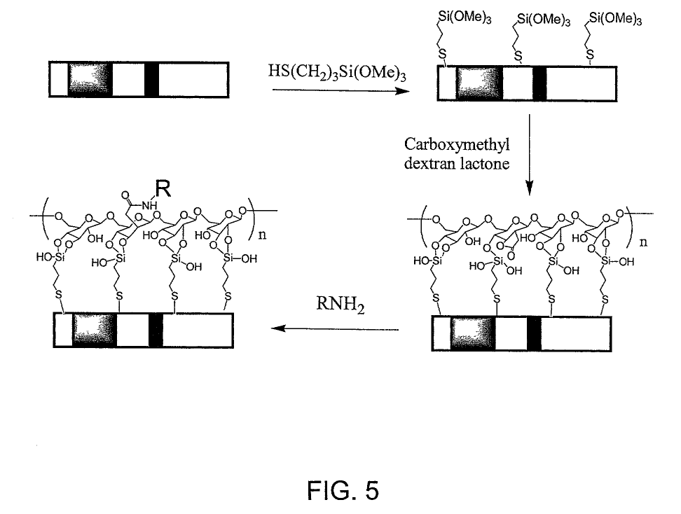

The initial derivatization of the nanoparticles can be accomplished with 3-mercapto propyl(trimethoxy)silane. Then, the silane alkoxy is exchanged with the free hydroxyls of a carboxymethyl dextran derived lactone. Subsequent cleavage of the lactone with amines carrying diverse functional groups will yield a library of gamma-hydroxy amides of dextran coated nanoparticles. These methods provide a common reactive intermediate that is easily prepared. This is illustrated in FIG. 5

The dextran-coated or hydrophilic SAMs simultaneously provide a surface that is resistant to non-specific interaction between the nanorod extraction probes and proteins having a wide range of molecular weights and isoelectric points. By appropriately choosing and designing structurally distinct amine reactants for derivatization, there is an opportunity to create a vast library of extraction phases. This may be prepared using a combinatorial process. These combinatorially-derivatized nanoparticles would present extraction phases with varying avidity for binding to the wide variety of molecules present in a biological sample. These will be expected to provide much greater efficacy than has been described for Surface Enhanced Laser Desorption Ionization Mass Spectrometry (SELDI-MS). Furthermore, compared to such protein “chip” technology, the use of segmented-nanoparticles as extraction probes will provide access to a greater number of different extraction phases. In addition, nanoparticle extraction probes will be able to achieve the interrogation using smaller sample volume, and will have a kinetic advantage (i.e., the small size of nanoparticles make interaction with biological sample almost homogenous compared to planar surface). In the protein “chip” technology, the protein probes are immobilized on a planar surface. Compared to the three-dimensional assortment of free nanoparticles extraction probes, a two-dimensional approach is hampered by a decrease in the efficiency of interaction and a greater degree of non-specific binding.

In contrast to systems based on “chips,” affinity capture techniques using nanoparticles extraction probes will use off-line incubation steps for capturing the analytes (i.e., the segmented-nanoparticle will go into the sample while the sample will go onto the “chip”). Using nanoparticles as the extraction probes is inherently superior from a kinetic viewpoint because it results in more rapid capture of analytes. In addition, this approach is advantageous from mass action perspective to drive binding—the density of the binding determinant (i.e., ligand or capture agent) on the nanoparticle can be varied to accommodate the wide range of analyte concentrations that are encountered in a biological fluid.

Carbohydrate derivatized SAMs with varying densities have been used to address issues involving cell-surface carbohydrate-protein interactions. These surfaces can be tailored to recognize free saccharides and, at the same time, are designed to take advantage of multiple binding determinants for carbohydrates in glycoproteins, for example. This approach will provide extraction phases capable of binding a wide spectrum of molecules, from low molecular weight organic compounds to large proteins, which are addressable and amenable to analysis.

Biological Marker Applications

The extraction probes and methods of the present invention can be used to obtain comprehensive, detailed information about a sample. This information may be used to phenotype a given organism or class or sub class of organisms. These phenotypes may be manipulated (e.g., by computational analysis) to identify a biological marker or to assess the effect of a perturbation in the organism.

The levels of a biological marker may vary widely from individual to individual. In many cases such variations may be random, but this may not always be the case. For example, in some situations, baseline levels may be individual specific, and only by taking multiple readings from an individual would it be possible to identify a biological marker. Although it may not be likely that a baseline would be established for a healthy individual, there may be valuable information gained from the variations over time in a given individual that has a disease or medical condition. For example, a patient with rheumatoid arthritis may show interesting variations when off or on medicine, or when exhibiting a severe flare-up of symptoms. If such longitudinal correlations exist, review of the longitudinal data of other similarly situated patients could confirm valuable biological markers associated with the disease.

Thus, the present invention encompasses a method for detecting analytes that are differentially present in a first sample and a second sample (e.g., normal/disease; treated/untreated; early/late, etc.). The method would typically proceed by having two sets of extraction probes which preferably contain substantially the same distribution of different extraction probes. The first set of extraction probes is contacted with the first sample and the second set of extraction probes is contacted with the second sample. After the probes have been given the opportunity to interact with the sample, the two sets are each separated from their respective samples, by any of a number of means known in the art, and analyzed as discussed above. Comparing the results of the two analyses provides critical information necessary to identify changed components, patterns, and so on. The differences observed between the two samples can be identified and further explored. The number of extraction probes used in a set will vary depending on the context and purpose of the invenstigation. Preferably, the set of analytes sets being compared comprise at least 10 analytes and, more preferably, at least 100 analytes. In this way, the present invention may be used to study normal biological functions, disease, disease progression, and changes associated with virtually any perturbance to the organism. Indeed, information the present invention makes available may be analyzed to identify biological markers that can be measured and evaluated as indicators of normal biological processes, pathogenic processes, or pharmacologic responses to a therapeutic intervention.

An additional application of the present invention is in monitoring dose response studies. In this application, a population of individuals is evaluated before and after the administration of a drug and after increasing doses of the drug. The selected population may be healthy individuals, and the anticipated biological dose response endpoint is toxicity or side effect profiles. Where the individuals have a particular disease or medical condition, markers may be identified for efficacy along with the negative effects of the drug. By evaluating the information from individuals before and after administration of drugs, it will be possible to identify markers or marker groupings associated with administration and response to the drug. In some situations, such markers could be used as an endpoint for clinical studies. For example, in contrast to such clinical endpoints as disease progression and recurrence or quality of life measures (which typically take a long time to assess), biological markers may provide a more rapid and quantative measure of a drug's clinical profile.

In other applications of the present invention, longitudinal studies of individuals receiving a drug or treatment for the prevention or treatment of a disease or medical condition could constitute the population of individuals being evaluated. By correlating biological indicators of individuals before they receive treatment with subsequent clinical observations, it will be possible to identify markers associated with those members of a potential patient population that will most benefit from the treatment therapy. In such a manner, expensive treatments can be limited to the subpopulations of patients most likely to benefit from the treatment.

Another application of the present invention is in the search for biological markers that identify patients who have early clinical signs of a disease. This would be extremely valuable for a multitude of disease states where a patient may have “subclinical” signs and symptoms which are not severe enough to bring the to the doctor's office. However, if a patient had a marker that was discovered in their blood, and they were advised to seek medical attention, their “subclinical” signs could be identified as their earliest phenotypic presentation of a disease. For many diseases, it is extremely advantageous to diagnose a disease as early as possible so that therapeutic drugs may be started and generally lead to reduced morbidity and mortality of that disease entity for the individual. A possible scenerio would be if a patient could take a blood test to see if they have a biological marker for Rheumatoid Arthritis. If the marker were present, they could the seek treatment during the “subclinical” stage where they may only have a sensation of warmth in their joints instead of waiting until they have pain, swelling and deformity. That individual would likely have a much better long-term outcome for Rheumatoid Arthritis in comparison to someone who waits until they have much later stage of the disease before seeking treatment.

See U.S. patent application Ser. No. 09/558,909, filed Apr. 26, 2000, entitled, “Phenotype and Biological Marker Identification System,” incorporated herein in its entirety by reference.

### EXAMPLES

### Example 1

Preparation Of Carboxy Terminated Dextran Coated Segmented-nanoparticles For Protein Conjugation

A solution of aminodextran (Molecular Probes, Eugene, Oreg.; 10 kD, 1 amine/10sugar residues) was made by dissolvingl 100 mg of the solid in 1 mL of phosphate buffer (pH 8.0, 10 mM). To this solution was added 375 μl of a 32 mM freshly made solution of SPDP (Pierce, Rockford, Ill.) in DMSO. The solution was vortexed and allowed to shake in an end over end shaker for 12 h. The SPDP derivatized aminodextran was then reduced with triscarboxyethyl phosphine (TCEP, Molecular Probes, Eugene, Oreg.) by adding 400 μL of a 32 mM TCEP solution. The mercapto aminodextran (80 mg/mL, 100 μL ) thus obtained was incubated with 2×10⁸ nanorods (Ag/Au/Ag stripes) with end over end shaking for 15 h. The particles were made to 1.0 ml by diluting with water, centrifuged at 14 k for 2 min, and the supernatant discarded. The particles were resuspended in pH 8.0 phosphate buffer (1.0 mL) by sonication and then washed by centrifugation at 14 k rpm.

The supernatant was discarded and the particles were subjected to a final resuspension, centrifugation followed by a final suspension in 100 μL of phosphate buffer (pH 8.0. 100 mM). A solution of succinic anhydride in DMSO (100 mg/mL) was prepared and added dropwise to the suspension of the dextran coated segmented-nanoparticles. The succinic anhydride solution was added in 10 μL aliquots followed by 10 μL aliquots of 1.0(M) NaOH between each addition. A total of 10 additions were made over a period of 30 minutes. The particles were washed by the usual centrifugation, removal of supernatant followed by resuspension for a total of 3×1 mL washes. The carboxy-terminated dextran coated segmented-nanoparticles were then stored in 100 μL of water. The presence of dextran was qualitatively determined by the anthrone test (Anal. Biochem, 68, 332-335 (1975)).

### Example 2

Coupling Of Streptavidin To Carboxy-Terminated Dextran Coated Segmented-nanoparticles

A suspension of the carboxy-terminated dextran coated segmented-nanoparticles in 100 μL of MES buffer (pH 6.1, 10 mM) was treated with 10 μL of sulfo N-hydroxysuccinimide (10 mg/mL, Pierce) followed by 10 μL of ethyl dimethylaminopropyl carbodiimide (10 mg/mL in H₂O, Pierce). The mixture was shaken in an end over end shaker for 30 minutes and then gradually added to 50 μl of a 5 mg/mL solution of streptavidin in phosphate buffer (pH 8.0, 100 mM). The solution pH was adjusted to 8.0 with a few drops of 1.0M NaOH. The reaction mixture was shaken in an end over end shaker for 14 h. The volume was made to 1.0 mL with water and the segmented-nanoparticles washed by repeated centrifugation, removal of supernatant and resuspension in a fresh buffer for a total of three cycles. The streptavidin coated segmented-nanoparticles were stored in 100 μL of water at a concentration of 2×10⁹ particles per ml. The streptavidin number per particle was determined from the depletion of fluorescence when different concentrations of particles were incubated with a fixed concentration of the biotin-fluorescein conjugate.

### Example 3

Preparation Of Oligonucleotide Coated Particles

The oligonucleotide particles were prepared by incubating biotinylated (dT)21 mer (0.3 nmoles, HPLC purified from IDT Inc) with streptavidinated particles (2×10⁸ particles, 1×10⁵ streptavidin per particle) in a TRIS/EDTA buffer (100 μL, pH 8.0). The particles and the oligonucleotides were shaken in an end over end shaker for 1 h, after which it was washed by centrifugation, removal of supernatant and resuspension in water for a total of three cycles.

### Example 4

Coupling Aminophenylboronic Acid To NHS-Linked 8.6 μm Beads.

NHS-Ester linked beads (10 mg) were taken up in pH 9, TAPS buffer in a 1.5 ml eppendorf tube. To the bead solution, 20 μl of aminophenylboronic acid (10 mg/ml) was added. The solution was rotated overnight. The beads were then centrifuged and the supernatant removed. The beads were then resuspended in fresh TAPS buffer and washed two more times before they were suspended to a final concentration of 50 mg/ml in TAPS buffer.

### Example 5

Preparation Of Streptavidin Coated Particles

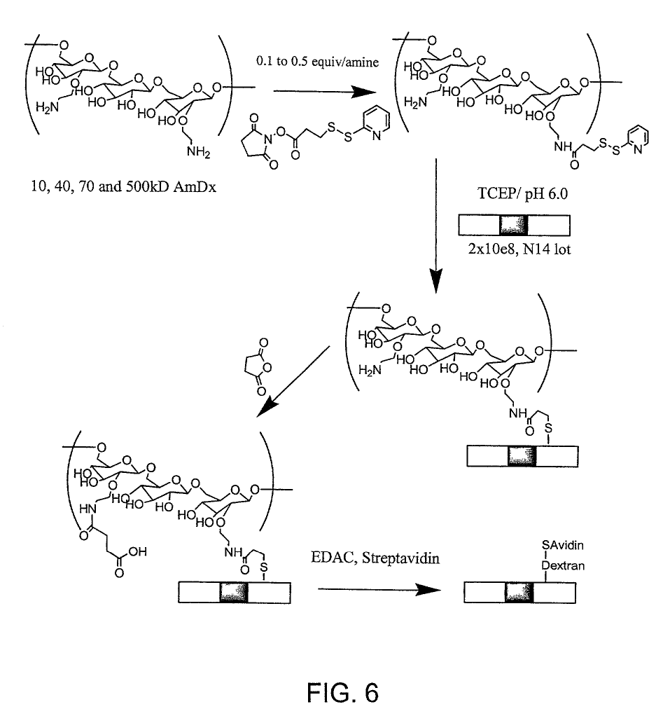

A suspension of carboxy modified latex particles (8.6 microns, Bangs lab) was constituted to 20 mg/ml in MES buffer (pH 6.0, 10 mM). To a 1.0 ml solution was added 10 μl of a freshly prepared solution of EDAC (10 mg/ml) in water. The reaction mixture was incubated in an end over end shaker for 15 minutes. The activated bead suspension was then added to a streptavidin solution (100 μl of 0.5 mg/ml) and the pH adjusted to 7.5 with phosphate buffer. The coupling reaction was allowed to proceed for 2 hour and then quenched with 1 mL of 1M glycine (pH 9.0). The particles were then washed by centrifugation at 5 K for 10 minutes, decanting the supernatant and resuspending in water (1 mL). The centrifugation, decantation and resuspension were repeated for a total of three cycles. This is illustrated in FIG. 6.

### Example 6

Alternative Preparation Of Streptavidin Coated Nanorods

SAM-coating of segmented-nanoparticles. A segmented-nanoparticle solution (10 μl, ˜1×10⁸ rods/ml) was added to 500 μl of SAM solution (@100 mM in ethanol, freshly made). The solution was incubated overnight using either a stir bar on a stirring plate or the rotator. Then the solution was rinsed with 200 μl of ethanol and H₂O mixtures at ratios of 1:0, 3:1, 1:1, 1:3, 0:1, respectively.

### Example 7

### Combinatorial Separation With Derivatized Particles

The following particles were used: 10 mg (8.6 microns, 3×10⁷ particles) of boronic acid derivatized magnetic particles in 100 μL of TAPS buffer, 00lig of streptavidinated particles (3×10⁵ particles, 10⁵ streptavidin per particle) in 100 μL of water and 10 mg of carboxy modified latex particles in 100 μL of water. The particles were added to a solution 15 μL of 1 mg/mL glucose +25 μL of 250 nM biotin-fluorescein (Molecular Probes) conjugate and 100 μL of 1 mM dioxadodecanediamine (Aldrich). The mixture was vortexed and allowed to shake in an end over end shaker for 12 h. The suspension was centrifuged and the supernatant analyzed for depletion of the analytes.

Biotin-Flourescein Conjugate Depletion Was Determined By Fluorescence (490 Excitation, 520 Emission) 58597 Units For Control And 3369 In Supernatant. Glucose depletion was determined by the anthrone test, (Anal. Biochem, 68, 332-335 (1975)) absorbance at 626 nm was 0.121 for control and 0.06 in the supernatant Diamine depletion was determined by the TNBS assay, absorbance at 415 nm was 0.620 for control and 0.332 in the supernatant. The control numbers in the above experiments refer to the analyte concentration measure before extraction, 300 μL of water was used instead of the particles.

### Example 8

Reacting Boronic Acid Linked Beads With Glucose Reacting Streptavidin Beads With Biotin-Fluorescein Conjugate

To a 1.5 ml Eppendorf tube, 20 μl of streptavidin linked bead solution (5 mg/ml, 10 ug) was added. To the same tube, 200 μl of boronic acid linked bead solution (50 mg/ml, 10 mg) in pH 9 TAPS buffer was also added. To the dual bead solution, 15 μl of glucose solution (1 mg/ml) was added, and 25 μl of biotin/fluorescein solution (250 nM) was added. The tube was rotated for approximately two hours. The beads were then centrifuged and the supernatant was removed from the precipitated beads.

The supernatant was then tested for fluorescein depletion and glucose depletion. For fluorescein, compared to a standard solution representing 0% depletion (58597 units), the experimental value for the supernatant indicated over 95% depletion of fluorescein (3369 unit). For glucose, compared to a standard solution representing 0% depletion (A=0.121), the experimental value for the supernatant indicated 30% depletion (A=0.110) (The standard and experimental glucose solutions were increased in concentration by a factor of 3, so a 10% decrease in absorbance is really representative of 30% depletion). This is illustrated in FIG. 6.

### Example 9

Preparation of SAM-coated Nanoparticles

Nanoparticles with four different surface functionalities (carboxyl-terminated, amine-terminated; sulfonate-terminated, and alkyl) were generated.

First, 1 ml each of 5 different 50 mM thiol solutions were prepared. To 4 different 1.5 ml microcentrifuge tubes, the following were added: 14.4 mg of 1-octadecanethiol; 11.0 mg of 11-mercaptoundecanoic acid; 9.0 mg of 3-mercapto-1-propanesulfonic acid; and 5.6 mg of 2-aminoethenthiol hydrochloride. The thiol samples were then dissolved in ethanol. To the 1-octadecanethiol, 11-mercaptoundecanoic acid, and 2-aminoethenthiol, 200 proof ethanol was added. A 3:1 ratio of ethanol and doubly deionized water was added to the 3-mercapto-1-propanesulfonic acid. All of the tubes were vortexed until the thiols dissolved.

Using the prepared solutions, nanoparticles were generated with different functionalities. First, cylindrical gold nanoparticles of average length 4 mm were washed in ethanol. 1 ml of nanoparticles in ethanol were centrifuged in a 1.5 ml centrifuge tube at 10,000 rpm for 2 minutes. The supernatant was removed and 1 ml of 200 proof ethanol was added to the tube. The tube was sonicated for 30 seconds and vortexed and then centrifuged again at 10,000 rpm for 2 minutes. The supernatant was removed and the ethanol wash repeated. Finally, the nanoparticles were resuspended in 1 ml of 200 proof ethanol by sonication.

Next, 100 microliters of the suspended nanoparticles, at approximately 2×10⁹ rods/ml, were transferred to four 1.5 ml microcentrifuge tubes. One ml of each of the thiol solutions was added to one of the tubes, and the tubes were vortexed and sonicated for 30 seconds. The tubes were then placed on a rotator for gentle mixing overnight, and sonicated and vortexed once per hour in the initial three hours. The next morning, the coated nanoparticles were either used immediately or stored in solution at 4° C. The quality of quantity of nanoparticles were checked with an optical microscope.

### Example 10

SPNE-MS on Human Plasma and Serum (<10 kD Fraction) Using Prepared SAM-Coated Nanoparticles

The nanoparticles generated as described in Example 1 were first processed in preparation for SPNE. 100 microliters of the suspended amine, sulfonate, and carboxyl-coated particles were added to each of three 1.5 ml microcentrifuge tubes. For the C₁₈-coated particles, 200 microliters were used. To a fifth tube, 200 microliters of bare gold nanoparticles were added. The five tubes were centrifuged at 10,000 rpm for two minutes, and the supernatant was removed by pipetting. 1 ml of 200 proof ethanol was added to each tube, and the tubes were then vortexed and centrifuged at 10,000 rpm for 2 minutes. The supernatant was again removed, 1 ml of 3:1 ethanol and water was added to each tube, and the tubes were sonicated for 30 seconds and vortexed to suspend the particles completely. The tubes were again centrifuged and the steps repeated for 500 microliters of 1:1 ethanol and water and then 500 microliters of 1:3 ethanol and water. Finally, 500 microliters of desired buffer was added to each tube. To the carboxyl and sulfonate surfaces, 10 mM MES (pH 5.8) was added. To the amine surfaces, 10 mM NH₄HCO₃ (pH 8.5) was added. To the bare gold and C₁₈ surfaces, 0.1% TFA was added. The tubes were again centrifuged at 10,000 rpm for two minutes, the supernatant removed, and 100 microliters of the respective buffer added to each tube.

Next, the plasma and serum samples were prepared for extraction. Three different samples were prepared from each of the plasma and serum. The pH of the first sample was adjusted to 5.5 by adding 4.5 microliters of 0.3 N HCl to 50 microliters of sample. For the second sample, 2 microliters of 2.5% TFA were added to 50 microliters of sample to generate 0.1% TFA-containing samples. An additional 25 microliters of sample was used as is (at pH 8.5). 25 microliters of each sample was added to 1.5 ml microcentrifuge tubes. To four tubes of pH 5.5 samples (two plasma and two serum), 3×10⁶ carboxyl- or sulfonate-coated nanoparticles were added. Amine particles were added to the pH 8.5 samples, and C₈ and bare gold particles were added to the 0.1% TFA samples. All tubes were sonicated for 30 seconds and placed on a rotator for one hour. The supernatant was removed, 25 microliters of respective buffer was added to each tube, and the tubes were sonicated for 30 seconds, vortexed briefly, and centrifuged at 10,000 rpm for two minutes. The supernatant was removed, and 2 microliters of 10 mg/ml α-cyano-4-hydroxy cinnamic acid in 50% acetontirile/0.1% TFA solution were added to the tubes with carboxyl, sulfonate, and amine surfaces. 4 microliters of 10 mg/ml α-cyano-4-hydroxy cinnamic acid in 50% acetonitrile/0.1% TFA solution was added to tubes with bare gold and C₁₈ surfaces. The tubes were again vortexed and sonicated for 30 seconds to suspend the particles.

The extraction efficiency of the nanoparticles was determined by MALDI-TOF analysis using a 2×96-well plate. MALDI was carried out in the semi-automatic mode, with carboxyl, sulfonate, and amine surfaces being automatic, while gold and C₁₈ required operator attention. Calmix 2 was deposited automatically onto the plate, and 0.5 microliters each of carboxyl, sulfonate, and amine particles were deposited manually on two spots each. 1 microliter each of gold and C₁₈ particles were deposited manually on two spots each. The MALDI plate was air-dried for ten minutes, checked, and inserted into the machine. The spectra were then acquired for a mass range of 800-10,000 Da. The foregoing examples are presented as illustrations and should in no way be considered as limiting the scope of this disclosure.

What is claimed is:

1. A method for extracting a plurality of analytes from a sample, comprising:
providing at least 100 differentiable extraction probes capable of adsorbing analytes, each differentiable extraction probe comprising a solid support and a different extraction phase;
contacting said differentiable extraction probes with a sample suspected of comprising at least one of said analytes;
separating said differentiable extraction probes from said sample; and
distinguishing among said differentiable extraction probes.

2. The method of claim 1, wherein said differentiable extraction probes are encoded and distinguished in dependence on said encoding.

3. The method of claim 2, wherein said solid supports are encoded.

4. The method of claim 1, wherein at least 150 differentiable extraction probes are provided.

5. The method of claim 1, wherein at least 200 differentiable extraction probes are provided.

6. The method of claim 1, wherein at least 500 differentiable extraction probes are provided.

7. The method of claim 1, wherein at least 1000 differentiable extraction probes are provided.

8. The method of claim 1, wherein at least 10,000 differentiable extraction probes are provided.

9. The method of claim 1, wherein said extraction probes are contacted with said sample simultaneously.

10. A method for extracting a plurality of analytes from a sample, comprising:
providing a plurality of different extraction probes capable of adsorbing analytes, each different extraction probe comprising a nanoparticle and a different extraction phase;
contacting said extraction probes with a sample suspected of comprising at least one of said analytes; and
separating said extraction probes from said sample.

11. The method of claim 10, wherein said nanoparticles are segmented nanoparticles.

12. The method of claim 10 wherein said extraction probes are differentiable, and wherein said method further comprises distinguishing between at least two different separated extraction probes.

13. The method of claim 12 wherein said extraction probes are encoded, and wherein said separated extraction probes are distinguished in dependence on said encoding.

14. The method of claim 12 wherein said separated extraction probes are distinguished by an optical method.

15. The method of claim 14 wherein said separated extraction probes are distinguished by a method selected from the group consisting of absorbance, fluorescence, Raman, hyperRaman, Rayleigh scattering, hyperRayleigh scattering, CARS, sum frequency generation, degenerate four wave mixing, forward light scattering, back scattering, and angular light scattering.

16. The method of claim 12 wherein said separated extraction probes are distinguished by a method selected from the group consisting of near field scanning optical microscopy, atomic force microscopy, scanning tunneling microscopy, chemical force microscopy, lateral force microscopy, transmission electron microscopy, scanning electron microscopy, field emission scanning electron microscopy, electrical methods, mechanical methods, magnetic detection methods, and SQUID.

17. The method of claim 10 further comprising detecting at least one analyte associated with said separated extraction probes.

18. The method of claim 17 wherein said detecting step comprises quantifying said associated analyte.

19. The method of claim 17 wherein said detecting step comprises identifying said associated analyte.

20. The method of claim 10 wherein said extraction phase is selected from the group consisting of hydrophobic materials, hydrophilic materials, acids, bases, polyclonal antibodies, monoclonal antibodies, aptamers, small molecule receptors, polymers, molecular solids, non-molecular solids, metals, metal ions, cations, and anions.

21. The method of claim 10 wherein at least one of said extraction phases is selected from the group consisting of a protein, peptide, and nucleic acid, and wherein said at least one extraction phase interacts with an analyte selected from the group consisting of a protein, peptide, and nucleic acid.

22. The method of claim 10, wherein providing a plurality of different extraction probes comprises providing at least 10 different extraction probes.

23. The method of claim 22, wherein providing a plurality of different extraction probes comprises providing at least 100 different extraction probes.

24. The method of claim 23, wherein providing a plurality of different extraction probes comprises providing at least 1000 different extraction probes.

25. The method of claim 24, wherein providing a plurality of different extraction probes comprises providing at least 10,000 different extraction probes.

26. The method of claim 10, wherein said extraction probes are contacted with said sample simultaneously.

27. A method for extracting a plurality of analytes from a sample, comprising:
providing a plurality of differentiable extraction probes of different masses, each comprising a solid support and a different extraction phase and being capable of adsorbing an analyte;
contacting said extraction probes with a sample suspected of comprising at least one of said analytes;
separating said extraction probes from said sample; and
distinguishing among said differentiable extraction probes in dependence on said masses.

28. A method for extracting a plurality of analytes from a sample, comprising:
providing a plurality of different extraction probes encoded with spatially-resolvable codes, each extraction probe comprising a solid support and a different extraction phase and being capable of adsorbing an analyte;
contacting said extraction probes with a sample suspected of comprising at least one of said analytes;
separating said extraction probes from said sample; and
distinguishing among said different extraction probes in dependence on said spatially-resolvable codes.

29. The method of claim 28, wherein said codes are distinguished optically.

30. The method of claim 28, wherein said codes comprise spatially-resolvable reflectivities.

31. A method for extracting a plurality of analytes from a sample, comprising:
providing a position-addressable array of extraction probes, each comprising a solid support and a different extraction phase;
providing an array of capillary tubes addressable by said array of extraction probes, said capillary tubes containing sample aliquots;
contacting said array of extraction probes with said array of capillary tubes such that said extraction probes are positioned within said capillary tubes; and
separating said array of extraction probes from said array of capillary tubes.

32. The method of claim 31 wherein each extraction probe comprises a different extraction phase.

33. The method of claim 31 wherein each sample aliquot is different.

34. A method for extracting a plurality of analytes from a sample, comprising:
providing a position-addressable array of extraction probes, each comprising a fiber and an extraction phase, wherein each extraction probe is capable of adsorbing an analyte;
contacting said array of extraction probes with sample aliquots suspected of comprising at least one of said analytes; and
separating said array of extraction probes from said sample aliquots.

35. The method of claim 34 wherein each extraction probe comprises a different extraction phase.

36. The method of claim 34 wherein each sample aliquot is different.

37. The method of claim 34 wherein each fiber has a diameter of less than 100 microns.

38. The method of claim 37 wherein each fiber has a diameter of less than 1 micron.

39. A method for detecting analytes that are differentially present in a first sample and a second sample, said method comprising:
providing first and second sets of extraction probes capable of adsorbing different analytes, each extraction probe comprising a solid support and an extraction phase, wherein said first set and said second set contain a substantially equal distribution of different extraction probes;
contacting said first set of extraction probes with said first sample and said second set of extraction probes with said second sample;
separating said first set of extraction probes from said first sample and said second set of extraction probes from said second sample;
detecting a first analyte set associated with said first set of extraction probes and a second analyte set associated with said second set of extraction probes; and
comparing said first analyte set and said second analyte set.

40. The method of claim 39 further comprising identifying differences between said first analyte set and said second analyte set in dependence on said comparison.

41. The method of claim 39 wherein said first analyte set comprises at least ten analytes.

42. The method of claim 41 wherein said first analyte set comprises at least 100 analytes.

43. A method for detecting analyte isoforms in a sample, comprising:
providing a plurality of differently coded extraction probes, each comprising a solid support and a different extraction phase, wherein at least one of said extraction probes is capable of adsorbing a parent analyte and an isoform of said parent analyte;
contacting said extraction probes with a sample suspected of comprising said parent analyte and said isoform;
separating said extraction probes from said sample; and
detecting said parent analyte and said isoform in said separated extraction probes, wherein said parent analyte and said isoform are associated with extraction probes having the same code.

44. The method of claim 43, wherein said parent analyte is a parent protein and said isoform is a corresponding post-translationally modified protein.

45. The method of claim 43, wherein said extraction phase comprises a polyclonal antibody.

46. The method of claim 43, further comprising identifying said parent analyte and said isoform associated with said extraction probes.

47. The method of claim 46, wherein said parent analyte and said isoform are identified by mass spectrometry.

48. The method of claim 43, further comprising quantifying said parent analyte and said isoform associated with said extraction probes.

49. A method for designing analyte extraction probes, comprising:
providing a plurality of different extraction probes, each comprising a solid support and a different combinatorially-derived extraction phase, wherein each extraction probe is capable of adsorbing an analyte;
contacting said extraction probes with a sample suspected of comprising at least one of said analytes;
separating said extraction probes from said sample; and
identifying separated extraction probes that satisfy at least one predetermined extraction probe criterion.

50. The method of claim 49 wherein said extraction probe criterion comprises extracting at least one analyte of interest from said sample.

51. The method of claim 49 wherein said extraction probe criterion comprises extracting non-overlapping classes of analytes from said sample.

52. The method of claim 49 wherein providing a plurality of different extraction probes comprises providing between 4 and 100,000 different extraction probes.

53. The method of claim 52 wherein providing a plurality of different extraction probes comprises providing between 10 and 1000 different extraction probes.

54. The method of claim 49 wherein identifying said separated extraction probes comprises identifying between 10 and 50 separated extraction probes.

55. A method for extracting a plurality of analytes from a sample, comprising:
providing a plurality of different extraction probes capable of adsorbing analytes, each extraction probe comprising a solid support and a different combinatorially-derived extraction phase;
contacting said extraction probes with a sample suspected of comprising at least one of said analytes; and
separating said extraction probes from said sample.

56. The method of claim 55 wherein extraction phases of different extraction probes have different analyte specificities.

57. The method of claim 55 wherein at least one of said extraction phases has an affinity for one particular analyte.

58. The method of claim 55 wherein at least one of said extraction phases has an affinity for more than one particular analyte.

59. The method of claim 55 wherein at least one of said extraction phases comprises a polymer.

60. The method of claim 55 wherein at least one of said extraction phases comprises a self-assembled monolayer.

61. The method of claim 55 wherein said extraction phases comprise at least one material selected from the group consisting of a metal alloy, oxide, glass, ceramic, semiconductor, nucleic acid, oligonucleotide, carbohydrate, polysaccharide, peptide, protein, lipid, zeolite, and polyelectrolyte multilayer.

62. The method of claim 55 wherein said extraction phases are generated randomly.

63. The method of claim 55 wherein said extraction phases are selected from a combinatorial library.

64. The method of claim 55 further comprising detecting at least one analyte associated with said separated extraction probe.

65. The method of claim 64 wherein detecting said associated analyte comprises identifying said associated analyte.

66. The method of claim 65 wherein said associated analyte is identified using mass spectrometry.

67. The method of claim 64 wherein detecting said associated analyte comprises quantifying said associated analyte.

68. The method of claim 55 wherein providing a plurality of different extraction probes comprises providing between 4 and 100,000 different extraction probes.

69. The method of claim 68 wherein providing a plurality of different extraction probes comprises providing between 10 and 1000 different extraction probes.

70. The method of claim 55, wherein said extraction probes are contacted with said sample simultaneously.

71. The method of claim 55 wherein said solid support is a nanoparticle.

72. The method of claim 71 wherein said nanoparticle is a bead.

73. The method of claim 71 wherein said nanoparticle is an encoded nanoparticle.

74. The method of claim 73 wherein said encoded nanoparticle is a segmented nanoparticle.

75. The method of claim 55 wherein said solid support is a fiber.

76. A kit comprising at least 100 differentiable extraction probes capable of adsorbing analytes, each differentiable extraction probe comprising a solid support and a different extraction phase.

77. The kit of claim 76 wherein said solid supports are nanoparticles.

78. The kit of claim 77 wherein said solid supports are segmented nanoparticles.

79. The kit of claim 72 wherein said solid supports are fibers.

80. The kit of claim 72 wherein said extraction phases are combinatorially derived.

81. The kit of claim 72 wherein at least one of said extraction phases is a polymer.

82. The kit of claim 72 wherein at least one of said extraction phases is an antibody.

83. The kit of claim 72 wherein at least one of said extraction phases comprises a material selected from the group consisting of hydrophobic materials, hydrophilic materials, acids, bases, polyclonal antibodies, monoclonal antibodies, aptamers, small molecule receptors, polymers, molecular solids, non-molecular solids, metals, metal ions, cations, and anions.

84. The kit of claim 72 wherein at least one of said extraction phases comprises a material selected from the group consisting of a metal alloy, oxide, glass, ceramic, semiconductor, nucleic acid, oligonucleotide, carbohydrate, polysaccharide, peptide, protein, lipid, zeolite, and polyelectrolyte multilayer.

85. The kit of claim 72 wherein at least one of said extraction phases is a protein.

86. The kit of claim 72 wherein at least one of said extraction phases is a self-assembled monolayer.

87. The kit of claim 72 wherein said extraction probes are encoded.

88. The kit of claim 87 wherein said extraction probes are encoded by spatially-resolvable codes.
